SEQUENCE LISTING (10 sequences)

SEQ ID NO: 1 - DNA, 2534 bp, Homo sapiens
  CDS (124)..(1428)
gcagaggtgagtgccgggctcggcgctctgctcctggagctcccgcgggactgcctggggacagggactgctgtggcgctcggccctccactgcggacctctcctgagtgggtgcgccgagtcatggagggcgcagagctggccgggaagatcctttccacctggctgacgctggttctcggcttcatccttttaccttcggtcttcggagtgtctctgggcatctccgagatctacatgaagatcctagtgaaaactttagagtgggccacaatacgaattgaaaaaggaaccccaaaggagtcgattcttaaaaactctgcttctgttggtattatccaaagagatgagtcacccatggaaaaagggctctctggtctacgaggaagggactttgagctgtctgacgtgttttatttctccaagaagggattggaagccattgtagaagatgaagtgacccagaggttttcctcagaggagctagtgtcatggaatctcctcacaagaaccaatgtaaatttccagtacatcagtctgcggctcactatggtgtgggtgctgggcgtcatagtgcgctattgtgtcctactgcctctgagggttaccttggctttcattgggatcagtttgctggttataggaactacactggttgggcagctgccagacagcagcctcaaaaactggctgagtgaactggtccatctgacttgctgccggatctgtgtgcgagccctctctggtaccattcattatcataacaagcagtacagaccccagaagggaggcatttgtgttgccaaccatacttcccccattgatgttttaatcttgacaacggatggatgttatgctatggttggccaggttcatggcggcttgatgggaattattcagagagctatggtcaaggcttgtcctcatgtctggtttgaacgctcagaaatgaaggatcgacacctggttactaagagactaaaagaacatattgctgataagaagaaactacccatactaatttttcctgaaggaacttgcatcaacaatacttcagtcatgatgtttaaaaaggggagctttgaaattggaggaaccatacatccagttgcaattaagtataaccctcagttcggtgatgcattttggaacagtagtaaatacaacatggtgagctacctgcttcgaatgatgaccagctgggccatcgtctgtgacgtgtggtacatgccccccatgaccagagaggaaggagaagatgcagtccagtttgctaacagggttaagtctgctattgctatacaaggaggcctgactgaacttccctgggatggaggactaaagagagcaaaggtgaaggacatctttaaggaagagcagcagaaaaattacagcaagatgattgtgggcaatggatctctcagctaagaggacggatgacagcctttagatctagaactagcccttagaaatggaatggctttttttgttttgttttgttttattgttttgtttttattattgttaatcttttctacagaatgattgtctctacctctttatgccagaggcagaacctacaggtgccctttttggcttttgttgttgttgtaacattagccccatggattgtaaggtggtttactgagttaaaacagattctgcttttgtaaaatgatggcatcactgtggactgaatgaaatatttgtatagaaaaaagtgcttgaaaagtgtgtttggaactcatcgatagggtaattctccaaaaatgcccaaactctttttctgtaattagccttgccactttcttcagtcacttaaatggtgagattacacatcagtgcaagatgaccattatggttatggtctactgcaaggttgaaaggaaaaatggaggattgtatttaggaaaagggacaactttgtggccacctgctctgaaagtcaaaaggaaatgtaaattagtgtcattagtgtgttggaagagaaatactattcagtaagcttcgccaaagaaaagtgagtcaaagttaatgtgtgtgcgcatttatatgtaggcagctcgtagaccacattttaaccagcaactggtaacaaagagcttagttttccttgtttgaatgctgtagatctgtacctagtacccctcccatctactgatttgtttgtttttgtaaccaaacacattttcagatagaaggagccttaaaaaaaaaaaatcacattgagtaacttcagtatgaatgaatgagagtgtgtggagctacccctcaccctccacccctttgtgctttttattcccgaattttcccagtctcttaaacagaaaaatgactgatataattatcttttggaaactgagccttaattttttttagagggggaaataagttttccccaactcacacagcataagcaatgtttgacagcaatataatgccgttgtaaactactgagagtattgtatctgttctggtaaccatgtacagaatgtgaaactgtcttatgaatataaataaattctatatttcta

SEQ ID NO: 2 - protein, 434 aa, Homo sapiens
MEGAELAGKILSTWLTLVLGFILLPSVFGVSLGISEIYMKILVKTLEWATIRIEKGTPKESILKNSASVGIIQRDESPMEKGLSGLRGRDFELSDVFYFSKKGLEAIVEDEVTQRFSSEELVSWNLLTRTNVNFQYISLRLTMVWVLGVIVRYCVLLPLRVTLAFIGISLLVIGTTLVGQLPDSSLKNWLSELVHLTCCRICVRALSGTIHYHNKQYRPQKGGICVANHTSPIDVLILTTDGCYAMVGQVHGGLMGIIQRAMVKACPHVWFERSEMKDRHLVTKRLKEHIADKKKLPILIFPEGTCINNTSVMMFKKGSFEIGGTIHPVAIKYNPQFGDAFWNSSKYNMVSYLLRMMTSWAIVCDVWYMPPMTREEGEDAVQFANRVKSAIAIQGGLTELPWDGGLKRAKVKDIFKEEQQKNYSKMIVGNGSLS

SEQ ID NO: 3 - DNA, 2821 bp, Mus musculus
  CDS (220)..(1536)
gcatagcccgcagcccacgccgaggtgcagggaacctgcggggatcctcgctgccggagggcctcgagctgaagggaacgaggcgggcggcaaagactcctcagagcccggctccgggctggacgctgtgttttccgaactgcggctggactgcctgggactctgacacctacggctctcggctttcctggggacctcttcctgagggcgtgccgagacatggagggcgcagacctggcagtgaagctcctgtccacctggctgacgctggtgggcggcctcatccttttaccctcggccttcggattatccctgggtatctcggagatctacatgaagatcctggtgaaaaccttggagtgggccacattacgaattcagaaaggagcccccaaggagtcagctcttaaaaactcagcttctgtgggtattatccaaagagatgagtcacccatggagaaagggctctctggtcttcgaggaagggacttcgagctctctgatgtgttctatttctccaagaaggggttggaagccattgtggaggatgaagtgacccagaggttctcctccgaagagctagtatcatggaacctcctcacacgaaccaatgtcaactttcagtacatcagtccaaggctcaccatggtgtgggtgctgggtgtcctagtgcgctattgcttcctgctacctctgagggtcaccctggctttcattgggatcagcttgctgattataggaactacactggttggccagcttccagacagcagcctcaaaaactggctgagtgagctggtgcatctgacctgctgcaggatctgtgttcggtccctatctggcacgatccattaccataacaagcagtacagaccccagaagggaggtatctgtgtcgccaatcacacctctcccattgatgtcctaatcctggccaccgatggatgttacgccatggttgggcaggttcacggtggattgatggggatcattcagagagccatggttaaggcttgtcctcacgtctggtttgagcgctcagaaataaaggacagacatctagtgactaagagattaaaagaacacattgctgacaagaaaaagttacccattctaatcttcccagaaggtacttgcatcaacaatacttcagtcatgatgtttaaaaagggaagctttgaaatcggaggaaccatctatccagtggccataaagtataacccccagttcggcgatgccttctggaacagtagtaaatacaacttggtgagctacctgcttcgaatcatgaccagctgggccattgtctgcgacgtgtggtacatgcctcccatgaccagagaggaaggagaagacgcagttcagtttgcaaacagggttaaatctgctatcgctgtacaaggagggctgacggaacttccctgggatggagggctgaagagagcaaaggtgaaggacaccttcaaggaagaacaacagaagaattacagcaagatgatcgtgggcaacggatctcccaacctggcgagggactgaagcatcgtggataaacatggcttcgcagaactggccttcagaagtgaaagatgatgatgttgttttgttgtgtcgtttttttgcttgtttgtttattgttcatttttttttctacttgattttgtctctatcctcatgccagaagctgaacctgcaacaggtgtcctttttgatgttttcgttcttgtgttggctcaaggaattgtaaggcccgttcttgagtggaaacagacttttataaaatgatgatgttgatggtgattgaatgaaatctttgtatggagaaaaagtgcttctagaatatgtttggaatttgttaataagatacttctcaacaatgactccaaactctgtaatttgatttccctttctttaatctttttttatttttattattattgttattttttaaagacatggttttatttatgtagcccaggctggcctcaaactcactttgtagccaagactggctaagaactgactattctggctccacttcccacgtgctaggattacaggcaggtgcttccacacctaggtgtgacttcagtaacataccaggtgaggtcatcacaccatagtgcatttccattatggttacattctgctctcaggttgaaagaaaaatgggggattatttttagtgaaagaacaaatttgtggttatctgatctgaaaggaatggaatggagattatttttttctcaggccttggaggaccaggaccggttgctggctggccagattcatttaagaaccaggatgagtggtgttcattcccacaacacatctcattcagctgcagtggagaaagcactgttagcttttcttgtttgtatgctaaagatttcttttgtcttatgattgttgttttgttttgtttgtttgttttgtaacaaaatgaaattgtccaagggaaggagcctttaaaacaaaagaaccacactttctgcaggatacaagtgtgagctatgcggagcttgcctcctgtccaccctgcccccaacttctctgtagtggtttagccccaaactccccagccttgtcagcagaaaactaaatggtataactaccttttggaaaactgagccttaacattttttaaaggggaaacaagagttctctgagtccaatagcatgagctatatttgatagtgatctatttaatactgctgtgagctcttgaggctgttagctccggagcaaccctgtgtagaaagtgaactgtcttactgactatgaataaattttatatttttaaaaaaaaaaaaaaaaaaaaaaa

SEQ ID NO: 4 - protein, 438 aa, Mus musculus
MEGADLAVKLLSTWLTLVGGLILLPSAFGLSLGISEIYMKILVKTLEWATLRIQKGAPKESALKNSASVGIIQRDESPMEKGLSGLRGRDFELSDVFYFSKKGLEAIVEDEVTQRFSSEELVSWNLLTRTNVNFQYISPRLTMVWVLGVLVRYCFLLPLRVTLAFIGISLLIIGTTLVGQLPDSSLKNWLSELVHLTCCRICVRSLSGTIHYHNKQYRPQKGGICVANHTSPIDVLILATDGCYAMVGQVHGGLMGIIQRAMVKACPHVWFERSEIKDRHLVTKRLKEHIADKKKLPILIFPEGTCINNTSVMMFKKGSFEIGGTIYPVAIKYNPQFGDAFWNSSKYNLVSYLLRIMTSWAIVCDVWYMPPMTREEGEDAVQFANRVKSAIAVQGGLTELPWDGGLKRAKVKDTFKEEQQKNYSKMIVGNGSPNLARD

SEQ ID NO: 5 - DNA, 22 bp, Artificial
  mGPAT3 forward primer
cgtgctgagacatggagggcgc

SEQ ID NO: 6 - DNA, 22 bp, Artificial
  mGPAT3 reverse primer
agccatgtttatccacgatgct

SEQ ID NO: 7 - DNA, 22 bp, Artificial
  hGPAT3 forward primer
ctcctgagtgggtgcgccgagt

SEQ ID NO: 8 - DNA, 22 bp, Artificial
  hGPAT3 reverse primer
tgtcatccgtcctcttagctga

SEQ ID NO: 9 - DNA, 53 bp, Artificial
  FLAG-tagged mGPAT3 forward primer
ccaccatggactacaaagacgatgacgacaaggagggcgcagacctggcggtg

SEQ ID NO: 10 - DNA, 53 bp, Artificial
  FLAG-tagged hGPAT3 forward primer
ccaccatggactacaaagacgatgacgacaaggagggcgcagagctggccggg

(19) United States
(12) Patent Application Publication
(10) Pub. No.: US 2008/0038278 A1
(43) Pub. Date: Feb. 14, 2008

(54) GPAT3 encodes a mammalian, microsomal acyl-coa:glycerol 3- phosphate acyltransferase

(76) Inventors: Jingsong Cao, Needham, MA (US); Ruth E. Gimeno, Wellesley, MA (US); Jian-Liang Li, Gainesville, FL (US)

Correspondence Address: FITZPATRICK CELLA (WYETH), 30 ROCKEFELLER PLAZA, NEW YORK, NY, 10112-3800, US

(21) Appl. No.: 11/711,577

(22) Filed: Feb. 26, 2007

Related U.S. Application Data
(60) Provisional application No. 60/776,759, filed on Feb. 24, 2006.
(60) Provisional application No. 60/872,747, filed on Dec. 4, 2006.

(51) Int. Cl. A61K 39/395; A61K 31/7088; C07K 16/40; C12Q 1/48; C12N 5/00; A61K 38/00

(52) U.S. Cl. 424/158.1; 435/15; 435/375; 514/2; 514/44; 530/387.1; 530/387.9

(57) ABSTRACT

The present invention provides isolated and purified polynucleotides and polypeptides related to mouse and human microsomal acyl-CoA:glycerol 3-phosphate acyltransferase 3 (GPAT3) and their uses in modulating triacylglycerol (TAG) levels in a cell or sample of interest. The invention also provides GPAT3 agonists and antagonists, e.g., GPAT3 polynucleotides and polypeptides, antibodies to GPAT3 (agonistic and antagonistic antibodies), GPAT3 inhibitory polypeptides, and GPAT3 inhibitory polynucleotides. The present invention is also directed to novel methods for diagnosing, prognosing, monitoring, treating, ameliorating and/or preventing conditions related to GPAT3, TAG synthesis (or accumulation), or the synthesis (or accumulation) of TAG precursors (e.g., MAG, LPA, PA, and/or G3P). These GPAT3-associated conditions include, but are not limited to, dyslipidemia (e.g., hyperlipidemia, hypertriglyceridemia, Type III hyperlipidemia), obesity, hypercholesterolemia, hepatic steatosis, cancer, skin disorders associated with altered lipid metabolism (e.g., acne vulgaris, dry skin), adiposity, type 2 diabetes (and complications associated therewith, such as dermopathy, retinopathy, neuropathy, and nephropathy), insulin resistance, hyperinsulinemia, hypertension, cardiovascular disease, atherosclerosis, stroke, thrombosis, lipodystrophy, lipopenia, Reye's syndrome, Cushing's syndrome, metabolic syndrome (e.g., syndrome X), eating disorders (e.g., anorexia, bulimia), skin homeostasis, disorders related to energy storage, nutrient absorption, and lipid metabolism, reduced or absent lactation, and low preterm birth weight (and complications thereof, such as defects in neural development).

### RELATED APPLICATIONS

This application claims the benefit of priority from U.S. Provisional Patent Application No. 60/776,759, filed Feb. 24, 2006, and 60/872,747, filed Dec. 4, 2006, the contents of which are hereby incorporated by reference herein in their entireties.

### BACKGROUND OF THE INVENTION

1. Field of the Invention

The invention relates to a previously uncharacterized triacylglycerol biosynthetic enzyme, designated GPAT3 (e.g., mouse and human GPAT3), and active fragments thereof, as well as GPAT3 antagonists (e.g., inhibitory GPAT3 polynucleotides and polypeptides, antagonistic anti-GPAT3 antibodies, and inhibitory small molecules) that interfere with GPAT3 activity, and GPAT3 agonists (e.g., GPAT3 polynucleotides and polypeptides, agonistic anti-GPAT3 antibodies, and stimulatory small molecules) that enhance GPAT3 activity. In particular, the invention relates to mouse and human GPAT3 and related regulatory molecules, and their uses in regulating GPAT3-associated activities. The GPAT3 polynucleotides and polypeptides, and GPAT3 agonists and antagonists disclosed herein are useful in modulating triacylglycerol (TAG) synthesis and TAG accumulation, as well as in screening for compounds capable of modulating TAG synthesis and/or TAG accumulation. As such, the GPAT3 polynucleotides and polypeptides (including fragments and fusions thereof), and GPAT3 agonists and antagonists are predicted to be useful in diagnosing, prognosing, monitoring, preventing, and/or treating GPAT3-associated conditions, disorders associated with TAG metabolism (e.g., TAG synthesis, depletion, or accumulation) and/or disorders associated with TAG precursor metabolism (e.g., TAG precursor synthesis, depletion, or accumulation).

2. Related Background Art

Synthesis of triacylglycerols (TAGs) is a fundamental metabolic pathway critically important for energy storage, nutrient absorption, lactation, skin homeostasis, and transport and metabolism of free fatty acids (Coleman and Lee (2004) Prog. Lipid Res. 43:134-76; Lehner and Kuksis (1996) Prog. Lipid. Res. 35:169-201; Smith et al. (2000) Nat. Genet. 25:87-90; Stone et al. (2004) J. Biol. Chem. 279:11767-76). In animals, there are two main biochemical pathways for TAG biosynthesis: the monoacylglycerol (MAG) pathway and the glycerol 3-phosphate (G3P) pathway. The MAG pathway begins with the acylation of 1- or 2-MAG to TAG by acyl-CoA:monoacylglycerol acyltransferase (MGAT); this pathway plays a dominant role in intestinal TAG synthesis for fat absorption. The G3P pathway is a de novo triacylglycerol biosynthetic pathway found in most tissues. GPAT (acyl-CoA:glycerol 3-phosphate acyltransferase) catalyzes the first step in triacylglycerol synthesis by the G3P pathway, producing 1-acyl-glycerol 3-phosphate (acyl-G3P) or lysophosphatidic acid (LPA). Subsequently, LPA is acylated at the sn-2 position to form phosphatidic acid (PA) by acyl-CoA: 1-acyl-glycerol 3-phosphate acyltransferase (AGPAT), followed by a phosphohydrolyzation catalyzed by phosphatidic acid phosphatase (PTP) to form diacylglycerol (DAG). Both the MAG and G3P pathways share the final step of converting DAG to TAG, which is catalyzed by acyl-CoA:DAG acyltransferase (DGAT).

Enzymes in the triacylglycerol biosynthetic pathway are of considerable interest in the pathophysiology and treatment of disorders such as obesity, type 2 diabetes, dyslipidemia and atherosclerosis. For example, deletion of DGAT1 decreases body weight and improves insulin sensitivity in mouse models of obesity (Smith et al., supra; Chen and Farese (2005) Arterioscler. Thromb. Vasc. Biol. 25:482-86); modulation of DGAT2 in mice by antisense oligonucleotides improves hepatic steatosis and hyperlipidemia (Yu et al. (2005) Hepatology 42:362-71). Ablation of mitochondrial GPAT (GPAT1) in mice also leads to reduced fat pad mass, lower body weight, and lower hepatic VLDL secretion, as well as improved hepatic steatosis and insulin resistance (Hammond et al. (2002) Mol. Cell. Biol. 22:8204-14; Neschen et al. (2005) Cell Metab. 2:55-65). Since disorders such as obesity, type 2 diabetes, dyslipidemia, and atherosclerosis are closely associated with alterations in triacylglycerol and fatty acid synthesis, inhibition of enzymes in triacylglycerol synthesis is considered to be a means of treating these disorders (Chen and Farese (2005), supra; Chen and Farese (2000), supra; Subauste and Burant (2003) Curr. Drug Targets Immune Endocr. Metabol. Disord. 3:263-70; Shi and Bum (2004) Nat. Rev. Drug Discov. 3:695-710; Lewin et al. (2004) J. Biol. Chem. 279:13488-95).

GPAT catalyzes the initial and committed step of triacylglycerol de novo synthesis. In mammals, GPAT activity exists in multiple isoforms, which can be distinguished by subcellular localization (mitochondria vs. microsomes), sensitivity to N-ethylmaleimide (NEM), and substrate preference (Coleman and Lee, supra; Lehner and Kuksis, supra; Lewin et al., supra). The gene encoding mammalian mitochondrial NEM-resistant GPAT1 (mtGPAT1) was identified a decade ago, and found to play a key role in liver triacylglycerol synthesis (Hammond et al., supra; Neschen et al., supra; Yet et al. (1993) Biochemistry 32:9486-91). Early reports suggested that GPAT1-deficient mice have decreased adipose tissue mass (Hammond et al., supra); more recent studies have shown little effect of GPAT1 deficiency on the development of obesity (Neschen et al., supra). It is therefore likely that GPAT isoforms other than GPAT1 contribute to lipogenesis in adipose tissue.

Previously, the mammalian NEM-sensitive microsomal GPAT activity was demonstrated to account for 80% to 90% of total GPAT activity in most tissues, and 50% to 80% of total activity in liver (Coleman and Lee, supra). In contrast to a relatively modest increase in mitochondrial GPAT activity, the microsomal GPAT activity is dramatically induced during adipocyte differentiation (Yet et al., supra; Coleman et al. (1978) J. Biol. Chem. 253:7256-61), and is decreased in adipose tissue of rodent models of type 1 diabetes (Saggerson and Carpenter (1987) Biochem. J. 243:289-92).

Although a considerable number of attempts have been made to purify microsomal GPAT from various species (Kluytmans and Raju (1974) Prep. Biochem. 4:141-63; Yamashita and Numa, supra; Eccleston and Harwood (1995) Biochim Biophys. Acta 1257:1-10; Mishra and Kamisaka (2001) Biochem. J. 355:315-22), these attempts have not yielded the molecular makeup of the enzyme. Recently, identification of lipid biosynthetic enzymes has been facilitated by the availability of sequence information and identification of homologues or orthologues across different enzymes and species (Cao et al. (2004) J. Biol. Chem. 279:31727-34; Cao et al. (2003) J. Biol. Chem. 278:13860-66; Cases et al. (1998) Proc. Natl. Acad Sci. U.S.A. 95:13018-23; Cases et al. (2001) J. Biol. Chem. 276:38870-76; Yang et al. (2004) J. Biol. Chem. 279:55866-74; Yen and Farese (2003) J. Biol. Chem. 278:18532-37). A gene encoding an NEM-sensitive enzyme exhibiting both GPAT and dihydroxyacetone phosphate acyltransferase (DHAP-AT) activity was recently identified in yeast (Zheng and Zou (2001) J. Biol. Chem. 276:41710-16). This enzyme belongs to the same superfamily as GPAT1 (mtGPAT), making it likely that mammalian microsomal GPAT is also a member of this family. However, to date, no close mammalian homologs of this yeast gene have been identified.

### SUMMARY OF THE INVENTION

The present invention provides various methods and compositions related to a previously uncharacterized triacylglycerol biosynthetic enzyme, designated GPAT3. Thus in at least one embodiment, the invention provides a method for treating, ameliorating, or preventing a GPAT3-associated condition in a mammal comprising administering to the mammal a therapeutically effective amount of an agent that modulates the level of expression or activity of GPAT3 in the mammal, i.e., a GPAT3 antagonist or a GPAT3 agonist. In another embodiment, the agent is a GPAT3 antagonist selected from the group consisting of GPAT3 inhibitory polynucleotides or fragments thereof, GPAT3 inhibitory polypeptides or fragments thereof, antagonistic anti-GPAT3 antibodies, antagonistic anti-GPAT3 antibody fragments, and small molecules. In another embodiment, the agent is a GPAT3 agonist selected from the group consisting of GPAT3 polynucleotides or fragments thereof, polynucleotides that hybridize under high stringency conditions to a nucleic acid sequence or a fragment of a nucleic acid as sequence set forth in SEQ ID NO:1 or SEQ ID NO:3, GPAT3 polypeptides or fragments thereof, polypeptides encoded by a nucleic acid sequence or a fragment of a nucleic acid sequence as set forth in SEQ ID NO:1 or SEQ ID NO:3, polypeptides encoded by a nucleic acid that hybridizes under high stringency conditions to a nucleic acid sequence or a fragment of a nucleic acid sequence as set forth in SEQ ID NO:1 or SEQ ID NO:3, agonistic anti-GPAT3 antibodies, agonistic anti-GPAT3 antibody fragments, and small molecules. In a further embodiment, the GPAT3 associated condition is selected from the group consisting of dyslipidemia, obesity, hypercholesterolemia, hepatic steatosis, cancer, acne vulgaris, adiposity, type 2 diabetes, insulin resistance, hyperinsulinemia, hypertension, cardiovascular disease, atherosclerosis, stroke, thrombosis, lipodystrophy, lipopenia, Reye's syndrome, Cushing's syndrome, metabolic syndrome, anorexia, bulimia, reduced or absent lactation, and low preterm birth weight.

In another embodiment, the invention provides a pharmaceutical composition comprising a GPAT3 antagonist and a pharmaceutically acceptable carrier. In another embodiment, the GPAT3 antagonist is selected from the group consisting of GPAT3 inhibitory polynucleotides or fragments thereof, GPAT3 inhibitory polypeptides or fragments thereof, antagonistic anti-GPAT3 antibodies, antagonistic anti-GPAT3 antibody fragments, and small molecules. In another embodiment, the invention provides a pharmaceutical composition comprising a GPAT3 agonist and a pharmaceutically acceptable carrier. In another embodiment, the GPAT3 agonist is selected from the group consisting of GPAT3 polynucleotides or fragments thereof, polynucleotides that hybridize under high stringency conditions to a nucleic acid sequence or a fragment of a nucleic acid sequence as set forth in SEQ ID NO:1 or SEQ ID NO:3, GPAT3 polypeptides or fragments thereof, polypeptides encoded by a nucleic acid sequence or a fragment of a nucleic acid sequence as set forth in SEQ ID NO:1 or SEQ ID NO:3, polypeptides encoded by a nucleic acid that hybridizes under high stringency conditions to a nucleic acid sequence or a fragment of a nucleic acid sequence as set forth in SEQ ID NO:1 or SEQ ID NO:3, agonistic anti-GPAT3 antibodies, agonistic anti-GPAT3 antibody fragments, and small molecules.

In another embodiment, the invention provides an antibody or antibody fragment that specifically binds a GPAT3 polypeptide or a fragment of a GPAT3 polypeptide. In another embodiment, the GPAT3 polypeptide is a mouse GPAT3 polypeptide or a human GPAT3 polypeptide. In another embodiment, the GPAT3 polypeptide comprises the amino acid sequence set forth in SEQ ID NO:2 or SEQ ID NO:4. In other embodiments, the antibody antagonizes at least one GPAT3 activity. In other embodiments, the antibody agonizes at least one GPAT3 activity.

In another embodiment, the invention provides a method for decreasing TAG synthesis in a cell or cell population, comprising contacting a cell or cell population with a GPAT3 antagonist in an amount sufficient to decrease the level of expression or activity of GPAT3 in the cell or cell population, wherein the GPAT3 antagonist is selected from the group consisting of GPAT3 inhibitory polynucleotides or fragments thereof, GPAT3 inhibitory polypeptides or fragments thereof, antagonistic anti-GPAT3 antibodies, antagonistic anti-GPAT3 antibody fragments, and small molecules. In another embodiment, the invention provides a method for increasing TAG synthesis in a cell or cell population, comprising contacting a cell or cell population with a GPAT3 agonist in an amount sufficient to increase the level of expression or activity of GPAT3 in the cell or cell population, wherein the GPAT3 agonist is selected from the group consisting of GPAT3 polynucleotides or fragments thereof, polynucleotides that hybridize under high stringency conditions to a nucleic acid sequence or a fragment of a nucleic acid sequence as set forth in SEQ ID NO:1 or SEQ ID NO:3, GPAT3 polypeptides or fragments thereof, polypeptides encoded by a nucleic acid sequence or a fragment of a nucleic acid sequence as set forth in SEQ ID NO:1 or SEQ ID NO:3, polypeptides encoded by a nucleic acid that hybridizes under high stringency conditions to a nucleic acid sequence or a fragment of a nucleic acid sequence as set forth in SEQ ID NO:1 or SEQ ID NO:3, agonistic anti-GPAT3 antibodies, agonistic anti-GPAT3 antibody fragments, and small molecules.

In another embodiment, the invention provides a method for decreasing PA, LPA and/or DAG synthesis and/or accumulation in a cell or cell population, comprising contacting a cell or cell population with a GPAT3 antagonist in an amount sufficient to decrease the level of expression or activity of GPAT3 in the cell or cell population, wherein the antagonist is selected from the group consisting of GPAT3 inhibitory polynucleotides or fragments thereof, GPAT3 inhibitory polypeptides or fragments thereof, antagonistic anti-GPAT3 antibodies, antagonistic anti-GPAT3 antibody fragments, and small molecules. In another embodiment, the invention provides a method for increasing PA, LPA and/or DAG synthesis and/or accumulation in a cell or cell population, comprising contacting a cell or cell population with a GPAT3 agonist in an amount sufficient to increase the level of expression or activity of GPAT3 in the cell or cell population, wherein the agonist is selected from the group consisting of GPAT3 polynucleotides or fragments thereof, polynucleotides that hybridize under high stringency conditions to a nucleic acid sequence or a fragment of a nucleic acid sequence as set forth in SEQ ID NO:1 or SEQ ID NO:3, GPAT3 polypeptides or fragments thereof, polypeptides encoded by a nucleic acid sequence or a fragment of a nucleic acid sequence as set forth in SEQ ID NO: 1 or SEQ ID NO:3, polypeptides encoded by a nucleic acid that hybridizes under high stringency conditions to a nucleic acid sequence or a fragment of a nucleic acid sequence as set forth in SEQ ID NO:1 or SEQ ID NO:3, agonistic anti-GPAT3 antibodies, agonistic anti-GPAT3 antibody fragments, and small molecules.

In another embodiment, the invention provides a method for monitoring the course of a treatment of a GPAT3-associated condition in a patient, comprising (a) measuring the level of expression or activity of GPAT3 in a cell or sample of interest from the patient; (b) administering a GPAT3 antagonist to the patient; and (c) measuring the level of expression or activity of GPAT3 in a cell or sample of interest from the patient following administration of the GPAT3 antagonist, wherein a lower level of expression or activity of GPAT3 in the cell or sample of interest from the patient following administration of the GPAT3 antagonist, in comparison to the level of expression or activity of GPAT3 in the cell or sample of interest from the patient prior to administration of the GPAT3 antagonist, provides a positive indication of the treatment of the GPAT3-associated condition in the patient. In another embodiment, the invention provides a method for monitoring the course of a treatment of a GPAT3-associated condition in a patient, comprising (a) measuring the level of expression or activity of GPAT3 in a cell or sample of interest from the patient; (b) administering a GPAT3 agonist to the patient; and (c) measuring the level of expression or activity of GPAT3 in a cell or sample of interest from the patient following administration of the GPAT3 agonist, wherein a greater level of expression or activity of GPAT3 in the cell or sample of interest from the patient following administration of the GPAT3 agonist, in comparison to the level of expression or activity of GPAT3 in the cell or sample of interest from the patient prior to administration of the GPAT3 agonist, provides a positive indication of the treatment of the GPAT3-associated condition in the patient.

In another embodiment, the invention provides a method for prognosing a GPAT3-associated condition in a patient, comprising (a) measuring the level of expression or activity of GPAT3 in a cell or sample of interest from the patient at a first time point; and (b) measuring the level of expression or activity of GPAT3 in a cell or sample of interest from the patient at a second time point, wherein a lower level of expression or activity of GPAT3 in the cell or sample of interest from the patient at the second time point, in comparison to the level of expression or activity of GPAT3 in the cell or sample of interest from the patient at the first time point, indicates a decreased likelihood that the patient will develop a more severe form of the GPAT3-associated condition. In another embodiment, the invention provides a method for prognosing a GPAT3-associated condition in a patient, comprising (a) measuring the level of expression or activity of GPAT3 in a cell or sample of interest from the patient; and (b) comparing the level of expression or activity of GPAT3 in the cell or sample of interest to the level of expression or activity of GPAT3 in a reference cell or sample of interest, wherein a lower level of expression or activity of GPAT3 in the cell or sample of interest from the patient, in comparison to the level of expression or activity of GPAT3 in the reference cell or sample, indicates a decreased likelihood that the patient will develop a more severe form of the GPAT3-associated condition.

In another embodiment, the invention provides a method for prognosing a GPAT3-associated condition in a patient, comprising (a) measuring the level of expression or activity of GPAT3 in a cell or sample of interest from the patient at a first time point; and (b) measuring the level of expression or activity of GPAT3 in a cell or sample of interest from the patient at a second time point, wherein a greater level of expression or activity of GPAT3 in the cell or sample of interest from the patient at the second time point, in comparison to the level of expression or activity of GPAT3 in the cell or sample of interest from the patient at the first time point, indicates a decreased likelihood that the patient will develop a more severe form of the GPAT3-associated condition. In another embodiment, the invention provides a method for prognosing a GPAT3-associated condition in a patient, comprising (a) measuring the level of expression or activity of GPAT3 in a cell or sample of interest from the patient; and (b) comparing the level of expression or activity of GPAT3 in the cell or sample of interest to the level of expression or activity of GPAT3 in a reference cell or sample of interest, wherein a greater level of expression or activity of GPAT3 in the cell or sample of interest from the patient, in comparison to the level of expression or activity of GPAT3 in the reference cell or sample, indicates a decreased likelihood that the patient will develop a more severe form of the GPAT3-associated condition.

In another embodiment, the invention provides a method for monitoring a GPAT3-associated condition in a patient, comprising (a) measuring the level of expression or activity of GPAT3 in a cell or sample of interest from the patient at a first time point; and (b) measuring the level of expression or activity of GPAT3 in a cell or sample of interest from the patient at a second time point, wherein a lower level of expression or activity of GPAT3 in the cell or sample of interest from the patient at the second time point, in comparison to the level of expression or activity of GPAT3 in the cell or sample of interest from the patient at the first time point, provides an indication that the GPAT3-associated condition has decreased in severity. In another embodiment, the invention provides a method for monitoring a GPAT3-associated condition in a patient, comprising (a) measuring the level of expression or activity of GPAT3 in a cell or sample of interest from the patient; and (b) comparing the level of expression or activity of GPAT3 in the cell or sample of interest from the patient to the level of expression or activity of GPAT3 in a reference cell or sample of interest, wherein a lower level of expression or activity of GPAT3 in the cell or sample of interest from the patient, in comparison to the level of expression or activity of GPAT3 in the reference cell or sample, provides an indication that the GPAT3-associated condition has decreased in severity.

In another embodiment, the invention provides a method for monitoring a GPAT3-associated condition in a patient, comprising (a) measuring the level of expression or activity of GPAT3 in a cell or sample of interest from the patient at a first time point; and (b) measuring the level of expression or activity of GPAT3 in a cell or sample of interest from the patient at a second time point, wherein a greater level of expression or activity of GPAT3 in the cell or sample of interest from the patient at the second time point, in comparison to the level of expression or activity of GPAT3 in the cell or sample of interest from the patient at the first time point, provides an indication that the GPAT3-associated condition has decreased in severity. In another embodiment, the invention provides a method for monitoring a GPAT3 -associated condition in a patient, comprising (a) measuring the level of expression or activity of GPAT3 in a cell or sample of interest from the patient; and (b) comparing the level of expression or activity of GPAT3 in the cell or sample of interest from the patient to the level of expression or activity of GPAT3 in a reference cell or sample of interest, wherein a greater level of expression or activity of GPAT3 in the cell or sample of interest from the patient, in comparison to the level of expression or activity of GPAT3 in the reference cell or sample, provides an indication that the GPAT3-associated condition has decreased in severity.

In another embodiment, the invention provides a method of screening for a compound capable of antagonizing GPAT3 activity comprising the steps of: (a) contacting a sample containing GPAT3 with a compound of interest; and (b) determining whether the level of activity of GPAT3 in the contacted sample is decreased relative to the level of activity of GPAT3 in a sample not contacted with the compound, wherein a decrease in the level of activity of GPAT3 in the contacted sample identifies the compound as a compound that is capable of antagonizing GPAT3 activity. In another embodiment, a method of screening based on determining the levels of expression of GPAT3 is provided. In other embodiments, the invention provides methods of screening for compounds capable of agonizing GPAT3 activity based on determining levels of activity or expression of GPAT3.

### BRIEF DESCRIPTION OF THE DRAWINGS

FIG. 1A shows a sequence alignment and analysis of full-length human GPAT3 (hGPAT3) and mouse GPAT3 (mGPAT3). Two predicted transmembrane regions (TM1 and TM2) are indicated with bars above the sequences. Potential serine (S), threonine (T), and tyrosine (Y) phosphorylation sites are indicated in bold (as predicted by NetPhos 2.0 Server). The predicted N-glycosylation sites (N) are shown in bold (as predicted by NetNglyc Server). Once incorporated into the endoplasmic reticulum (ER) membrane, the stretch of amino acids between the TM domains is believed to be located on the lumenal side of the ER, while the acyltransferase domains are predicted to be located on the cytosolic side of the ER membrane. FIG. 1B shows an alignment of the conserved acyltransferase domains of hGPAT3 (amino acids 209-332), hAGPAT1(amino acids 83-211), and mitochondrial hGPAT1(amino acids 205-355); underlined segments labeled I-IV show conserved acyltransferase motifs. Based on a comparison with previously characterized glycerolipid acyltransferases, motifs II and III are thought to play a role in G3P binding, and motifs I and IV are thought to play a catalytic role.

FIGS. 2A-2G show enzymatic activity analysis of GPAT3 expressed in Sf9 cells. FIG. 2A shows an anti-FLAG Western analysis of N-terminally FLAG-tagged human DGAT1 (hDGAT1), native or N-terminally FLAG-tagged mouse GPAT3 (mGPAT3) and human GPAT3 (hGPAT3) expressed in Sf9 cells (uninfected cells—“wild type”). FIG. 2B shows human and mouse GPAT3 activity analyzed by butanol-extraction. (mean±SD, n=3). *,p<0.05 vs. wild type (uninfected cells) and hDGAT1-expressing cells. FIG. 2C shows thin layer chromatography (TLC) analysis of mouse and human GPAT3 (mGPAT3 and hGPAT3, respectively) activity in wild type Sf9 cells or cells infected with hDGAT1-, mGPAT3-, or hGPAT3-containing virus. GPAT activity was assessed by using either [¹⁴C]glycerol 3-phosphate ([¹⁴C]G3P, left panel) or [¹⁴C]lauroyl-CoA (right panel) as radiolabeled substrates. The embedded numbers represent the relative levels of formed radiolabeled LPA. Ori, origin of migration; LPA, lysophosphatidic acid; FFA, free fatty acid. The fast-migrating band appearing next to LPA may represent a G3P-dependent but acyl-CoA-independent product by endogenous enzyme(s), possibly phosphatidylglycerol phosphate or phosphatidylglycerol. Similar results were obtained in at least three independent experiments. FIG. 2D shows substrate concentration-dependence of GPAT activity of GPAT3 expressed in Sf9 cells. Assays were conducted with the indicated concentrations of [¹⁴C]G3P or lauroyl-CoA in the presence of 100 μM of lauroyl-CoA or [¹⁴C]G3P, respectively. Representative TLC images indicating the formation of LPA are shown at top, and the specific GPAT activities are shown below (mean±SD, n=3-4). In some experiments (FIG. 2E, right panel), lysates from mammalian HEK293 cells transfected with empty vector (vector), hGPAT3 -containing vector, or human mtGPAT1 (mtGPAT1)-containing vector were analyzed. FIG. 2E shows that GPAT activity conferred by GPAT3, but not mtGPAT1, is sensitive to NEM treatment. Cell lysates from Sf9 cells (left) and mammalian HEK293 cells (right) were preincubated with or without 0.4 mM NEM on ice for 15 min prior to assay. Data represent one of two independent experiments performed with similar result. FIG. 2F, GPAT activity using different acyl-CoA species as substrates: 150 μM [¹⁴C]G3P and 50 μM fatty acyl-CoA were used as substrates, products were visualized by TLC. Data represent the average of two independent experiments; variation between experiments was <15%. FIG. 2G, activity of GPAT3 toward different acyl acceptors: 25 μM [¹⁴C]Lauroyl-CoA and 200 μM of the indicated acyl acceptors were used. PA, phosphatidic acid; PG, phosphatidylglycerol; PS, phosphatidylserine; PC, phosphatidylcholine; DAG, diacylglycerol; TAG, triacylglycerol. Data are representative of two independent experiments with similar results.

Overexpression of human or mouse GPAT3 in mammalian cells leads to an increased incorporation of fatty acid into triacylglycerol (TAG) (FIG. 3A), but not into phospholipids (PE, PC, PS) (FIG. 3B). Metabolic labeling studies in HEK293 cells overexpressing human and mouse GPAT1 (hGPAT1, mGPAT3), human DGAT1 (hDGAT1) or human mtGPAT1 (hmtGPAT1) were performed as described herein. FIG. 3A, TLC analysis of the formation of neutral lipids. FIG. 3B, TLC analysis of the formation of polar lipids. The number beneath each band is presented as relative to the control level, which was arbitrarily assigned a value of 1. Control, empty vector. Data are representative of two independent experiments with similar results. PE, phosphatidylethanolamine; PS, phosphatidylserine; PC, phosphatidylcholine.

FIG. 4A provides a Western analysis of subcellular fractions from HEK293 cells overexpressing FLAG-hGPAT3. The number below each band is presented as relative to the level of expression in the lysates, which was arbitrarily assigned 1. FIG. 4B, TLC analysis of GPAT activity in subcellular fractions of HEK293 cells overexpressing FLAG-GPAT3 or mtGPAT1. FIG. 4C, Quantitative analysis of GPAT activity (mean±SD, n=3-4). *, P<0.05.

FIG. 5 shows tissue distribution of mouse (FIG. 5A) and human (FIG. 5B) GPAT3 mRNA detected by quantitative PCR (Q-PCR). Mouse and human cDNA panels generated from a variety of tissues were purchased from BD Biosciences Clontech, (Mountain View, Calif.), and Q-PCR was performed with gene-specific primer sets obtained from Applied Biosystems (Foster City, Calif.). Expression level of GPAT3 mRNA was normalized to 18S rRNA. Data are expressed as mean±S.D. (n=4). BAT, brown adipose tissue.

FIGS. 6A-6C show regulation of mGPAT3 mRNA expression and mGPAT3 activity in 3T3-L1 adipocytes. FIG. 6A shows the induction of mGPAT3 mRNA during 3T3-L1 differentiation. FIGS. 6B and 6C show siRNA-mediated knockdown (“GPAT3-siRNA”) of mGPAT3 in 3T3-L1 adipocytes in comparison to control siRNA (“Control”). mGPAT3 mRNA levels (FIG. 6B) and mGPAT3 activity (FIG. 6C) were determined by Q-PCR analysis and TLC separation as described in the Examples. Shown at top of FIG. 6C is representative TLC data showing the formation of LPA in the GPAT assay. Data are expressed as mean±S.D. (n=4). *, P<0.05.

FIGS. 7A-7C show regulation of mGPAT3 mRNA in mice. FIGS. 7A and 7B, mGPAT3 mRNA levels in white adipose tissue (“WAT”, FIG. 7A) or liver (“Liver”, FIG. 7B) of ob/ob mice compared to wild type control mice (“Cont.”). FIG. 7C, treatment of ob/ob mice with the PPARγ agonist rosiglitazone (“Rosi.”) increased mGPAT3 mRNA expression in WAT as compared to control mice (“Cont.”). Data are expressed as mean±S.E.M. (n=4). *, P<0.05.

FIG. 8 shows the tissue distribution of mouse (FIG. 8A) and human (FIG. 8B) mtGPAT1 mRNA detected by Q-PCR. Data are expressed as mean±S.D. (n=4). BAT, brown adipose tissue; Gas, gastrocnemius muscle; Ed. Fat, epididymal fat.

FIGS. 9A-9H show regulation of mtGPAT1 mRNA (upper panels) and DGAT1 mRNA (lower panels) during 3T3-LI differentiation (FIGS. 9A and 9E), in white adipose tissue (WAT, FIGS. 9B and 9F) or liver (FIGS. 9C and 9G) of ob/ob mice compared to wild type control mice, and in WAT upon treatment with rosiglitazone (Rosi.) (FIGS. 9D and 9H). Data are expressed as mean±S.E.M. (n=4). *, P<0.05.

### DETAILED DESCRIPTION OF THE INVENTION

The inventors have discovered that mouse and human GPAT3 (also referred to as “GPAT2” in U.S. Provisional Patent Application No. 60/776,759) are members of the acyltransferase family predominantly expressed in tissues characterized by active lipid metabolism, such as adipose tissue, small intestine, kidney, and heart (Example 5). The inventors have also shown that ectopic expression of mouse and human GPAT3 in insect cells leads to a significant increase in NEM-sensitive GPAT activity (Example 2), while acyltransferase activity towards a variety of other lysophospholipids and neutral lipid substrates is not altered (Examples 2 and 3). Further, the inventors have established that overexpression of mouse and human GPAT3 in mammalian cells results in increases in triacylglycerol levels, but not phospholipid formation (Example 3), and that GPAT3 is localized to the ER, as revealed by an immunocytofluorescence study in COS-7 cells overexpressing tagged GPAT3 (Example 4). Moreover, the inventors have established that, similar to other triacylglycerol biosynthetic enzymes, GPAT3 mRNA is dramatically upregulated during adipocyte differentiation, is downregulated in adipose tissue of ob/ob mice, and is upregulated upon treatment with a PPARγ agonist (Example 5). These findings identify GPAT3 as a new triacylglycerol biosynthetic enzyme. In addition, the inventors have identified the closest human (and mouse) homologue of GPAT3, i.e., AGPAT6 (also referred to as GPAT4). Accordingly, similar to other lipogenic enzymes, GPAT3 and AGPAT6 are believed to be useful as target(s) for the treatment of disorders related to alterations in triacylglycerol metabolism including, but not limited to, dyslipidemia, obesity, adiposity, type 2 diabetes (and complications associated therewith, such as dermopathy, retinopathy, neuropathy, and nephropathy), insulin resistance, hyperinsulinemia, hypertension, cardiovascular disease, atherosclerosis, stroke, lipodystrophy, Cushing's syndrome, metabolic syndrome (e.g., syndrome X), eating disorders (e.g., anorexia, bulimia), skin homeostasis, and disorders related to energy storage, nutrient absorption, lactation, and low preterm birth weight (and complications thereof, such as defects in neural development).

GPAT3 is closely related (generally 66% or greater identity across the entire molecule and 80% or greater identity within the acyltransferase domain) to the previously identified gene of unknown function known as LPAAT zeta or AGPAT6 (Li et al. (2003) J. Hum. Genet. 48:438-42) (also referred to as GPAT4). Specifically, an alignment of human GPAT3 (hGPAT3) with human AGPAT6 (hAGPAT6; GenBank accession number NM_{—}178819 (1-acylglycerol 3-phosphate 0-acyltransferase 6 (lysophosphatidic acid acyltransferase, zeta))) demonstrates that human AGPAT6 is 67% identical to human GPAT3 at the amino acid level overall, and 87% identical within the acyltransferase domain. Two recent reports also disclose phenotypes for AGPAT6-deficient mice (Beigneux et al. (2006) J. Lipid Res. 47:734-44; Vergnes et al. (2006) J. Lipid Res. 47:745-54); these reports confirm several findings disclosed herein, i.e., the high identity between GPAT3 and AGPAT6, the expression of GPAT3 at high levels in fat tissue and at low levels in liver, and essential roles in triacylglycerol synthesis of these two genes. Due to the high homology between GPAT3 and AGPAT6, it is also believed that AGPAT6 plays a fundamental role in disorders associated with TAG dysregulation, e.g., obesity, lipodystrophy and type 2 diabetes.

Here the inventors report the identification of mouse and human genes encoding microsomal proteins with GPAT activity (designated mGPAT3 and hGPAT3, respectively) by combination analysis of a glycerophosphate acyltransferase dataset and a transcriptional profiling dataset generated from differentiating adipocytes. The inventors demonstrate that overexpression of these genes confers NEM-sensitive G3P GPAT acyltransferase activity, and increases TAG formation in intact cells. The inventors also show that these genes are expressed and regulated in a manner suggesting an important role in lipogenesis, and that the mouse gene encodes a significant portion of GPAT activity in 3T3-L1 adipocytes. Thus, it is believed that microsomal GPAT (GPAT3) plays roles in both normal physiology and in pathological conditions, such as obesity and type 2 diabetes.

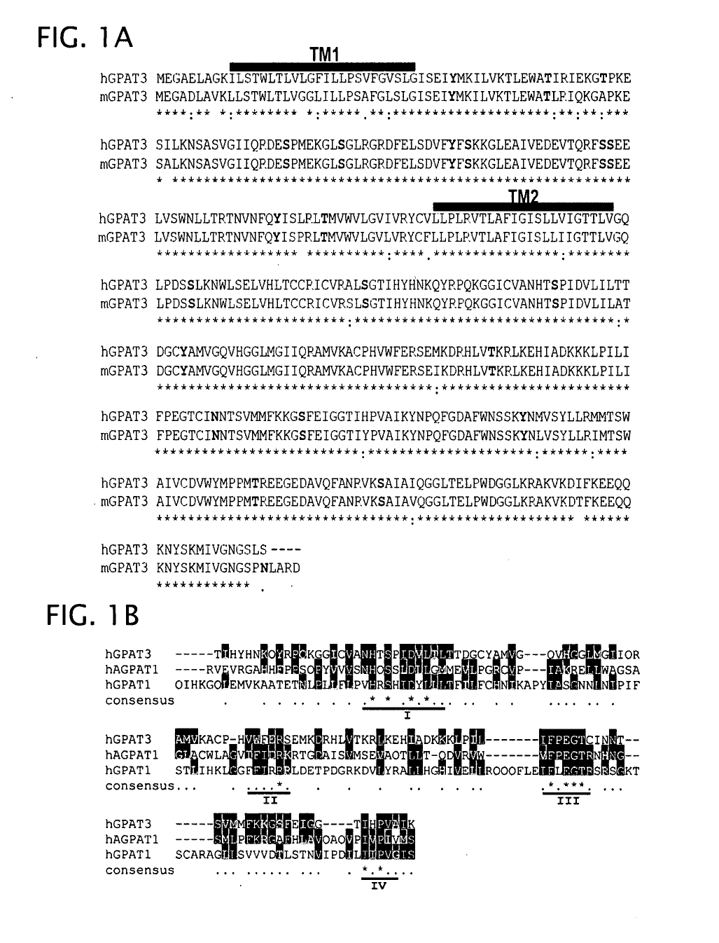

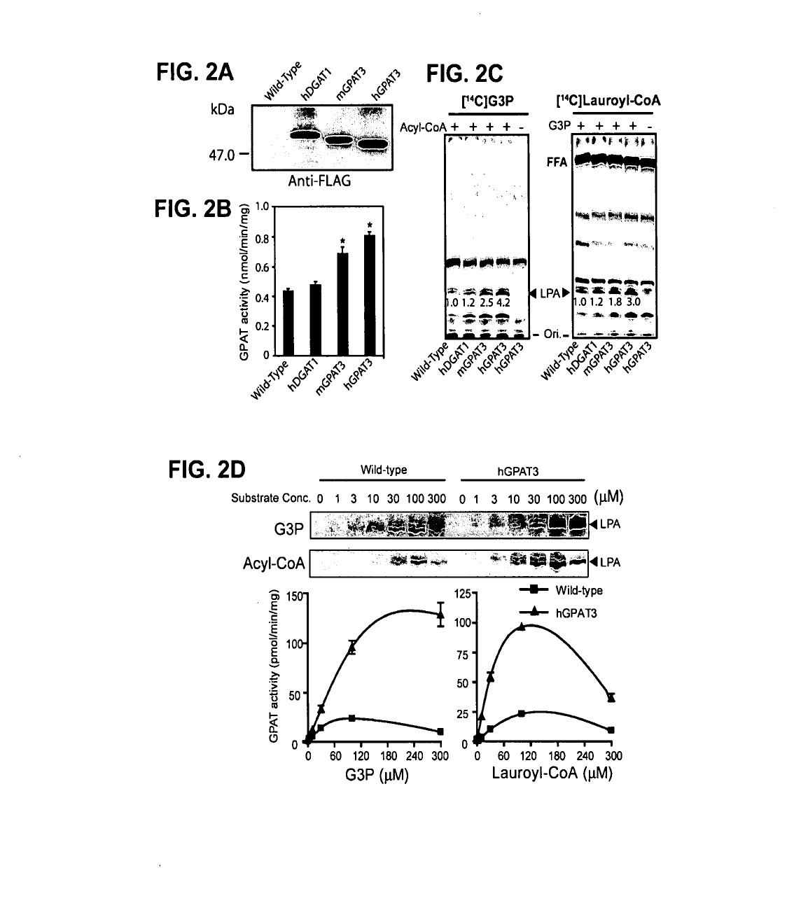

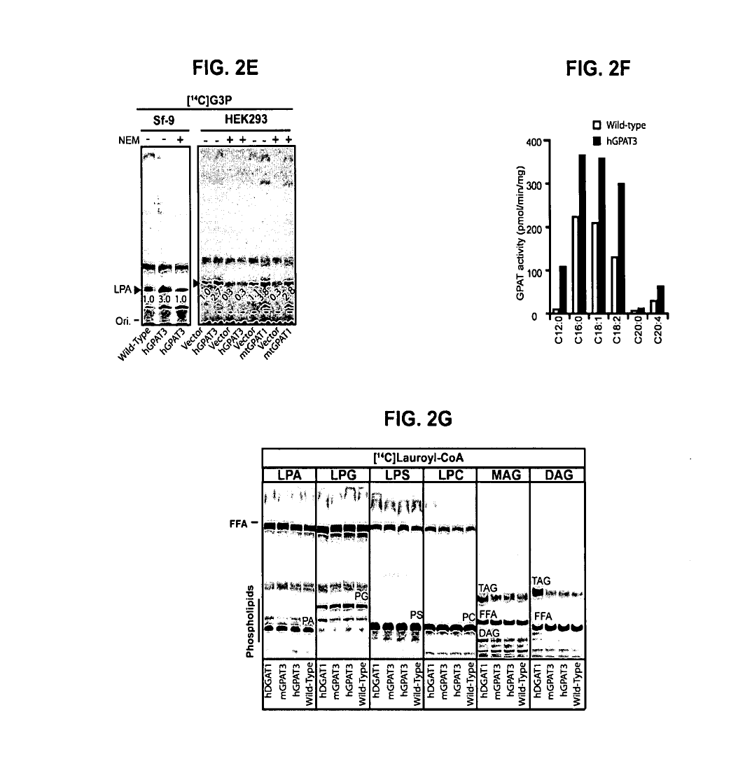

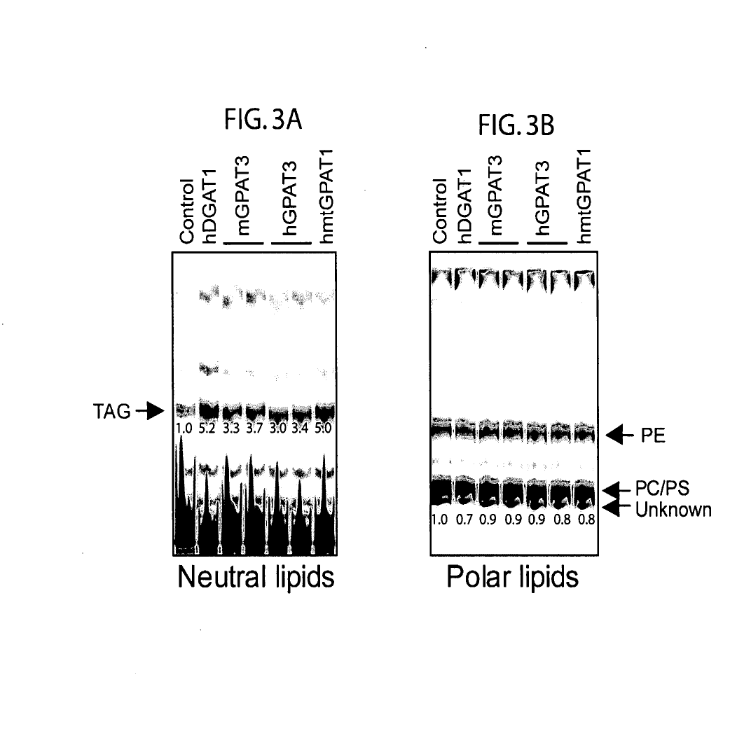

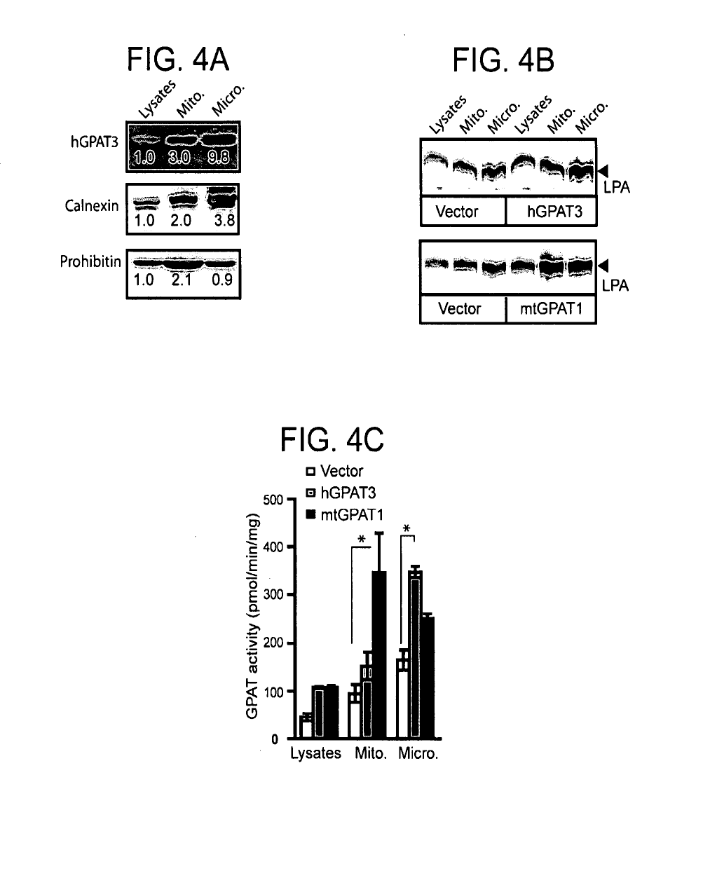

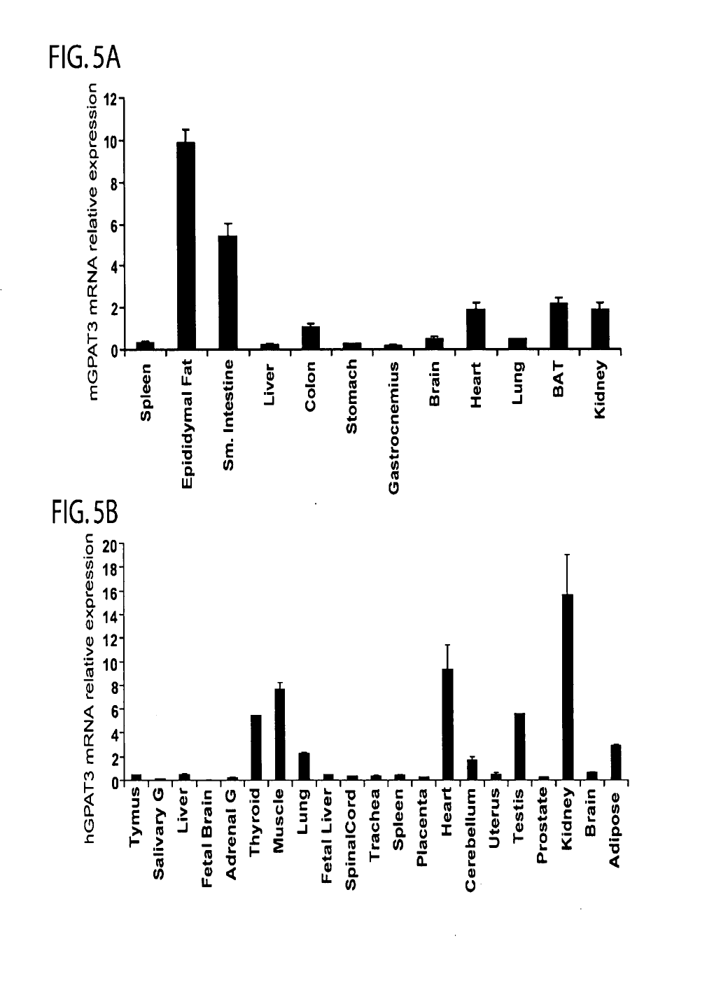

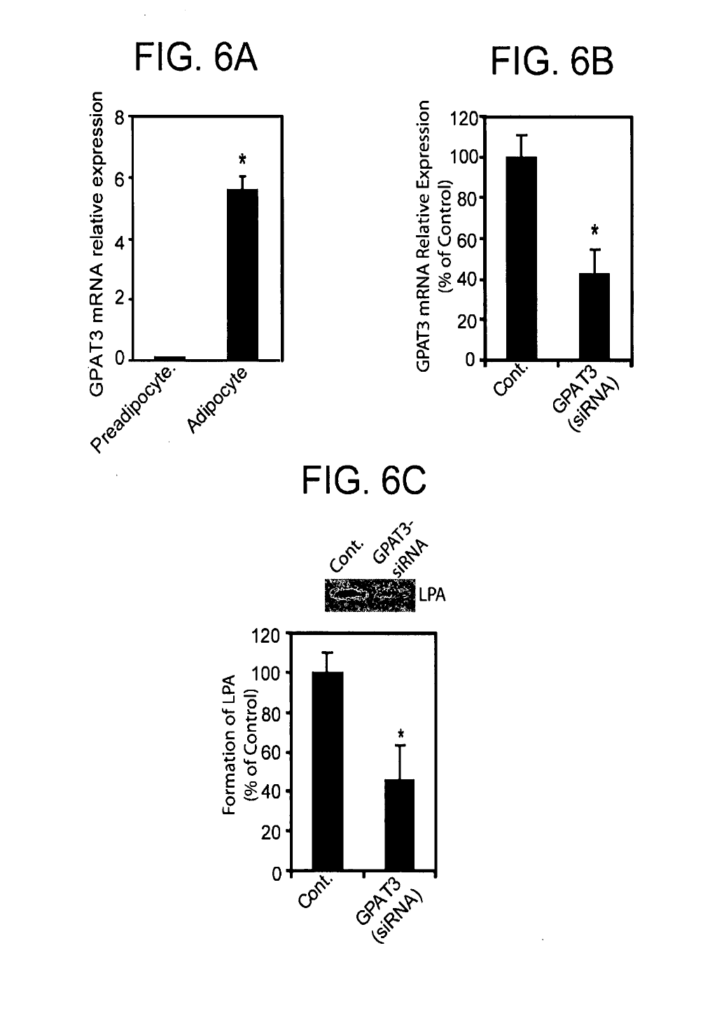

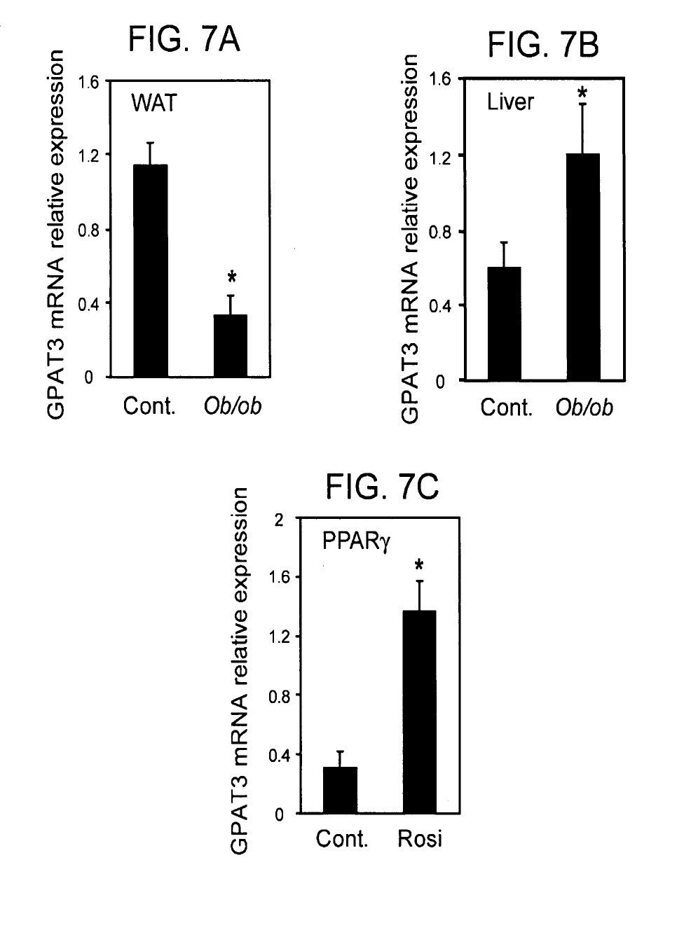

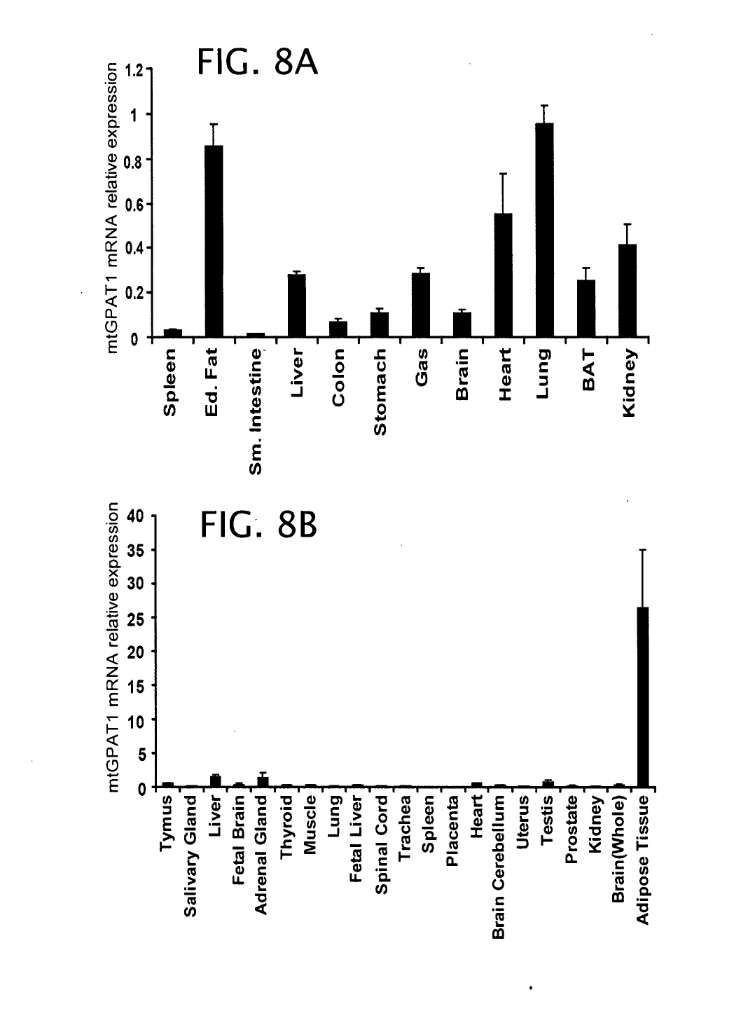

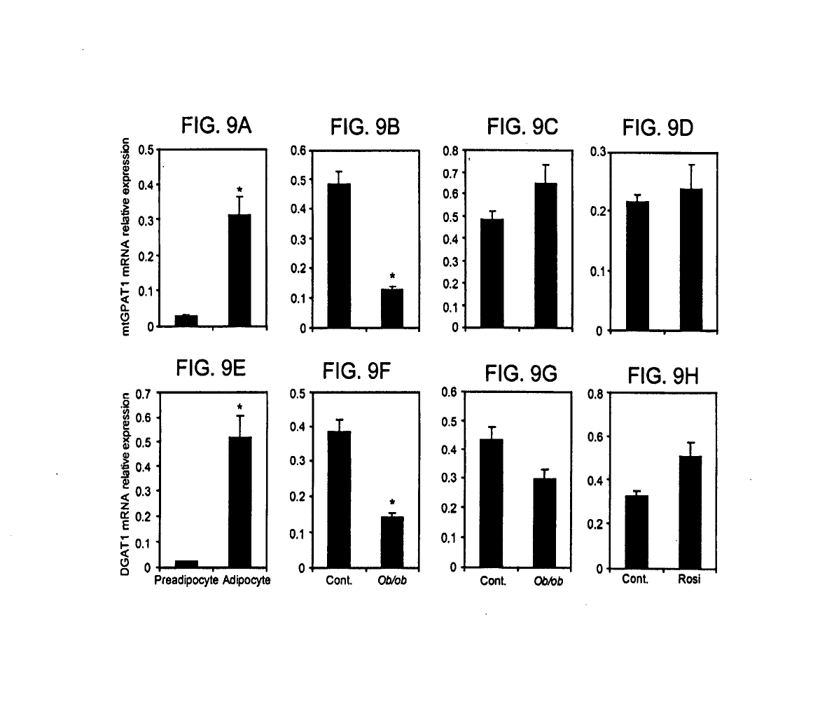

Several lines of evidence suggest that the human and mouse genes disclosed herein, i.e., the GPAT3 genes, encode a microsomal GPAT. First, GPAT3 contains all four conserved motifs that are found in most acyltransferases involved in glycerolipid metabolism (FIG. 1). Second, recombinant GPAT3 expressed in insect or mammalian cells exhibits NEM-sensitive GPAT activity (FIGS. 2 and 3). Third, expression of recombinant GPAT3 specifically increased CoA-dependent acylation of glycerol 3-phosphate, but did not alter acylation of lysophosphatidic acid, other lysophospholipids, monoacylglycerol or diacylglycerol (FIG. 2). Fourth, consistent with the reported broad substrate specificity of microsomal GPAT, recombinant GPAT3 enhanced LPA formation using a wide variety of long-chain-acyl-CoA as substrates, including both saturated and unsaturated acyl-CoA species (FIG. 2F). Fifth, GPAT3 localized to the ER upon overexpression in COS-7 cells, but was not observed in COS-7 mitochondria (FIG. 4). Sixth, GPAT3 mRNA was upregulated during 3T3-L1 adipocyte differentiation, consistent with the reported upregulation of microsomal GPAT activity in this cell line (FIG. 6A). Seventh, GPAT activity in differentiated 3T3-L1 adipocytes was significantly decreased by attenuating GPAT3 expression using siRNA, suggesting that GPAT3 accounts for a significant portion of GPAT activity in differentiated adipocytes (FIGS. 6B and 6C). These findings establish GPAT3 as a microsomal GPAT.

The product of the GPAT reaction, LPA, is an intermediate in both phospholipid and TAG synthesis. The present studies show that GPAT3 overexpression selectively increases TAG synthesis, but does not affect synthesis of phospholipids in the assays utilized (FIG. 2). A function in TAG synthesis for GPAT3 is further supported by the pattern of GPAT3 mRNA expression and regulation (FIG. 5). Similar to DGAT1 and mtGPAT (GPAT1), GPAT3 mRNA is highly expressed in white adipose tissue, is upregulated during adipocyte differentiation, and is downregulated in the adipose tissue of ob/ob mice. In addition, GPAT3 mRNA is increased in white adipose tissue of mice treated with the PPARγ agonist rosiglitazone (FIG. 7C), which is known to induce expression of lipogenic genes (e.g., Rosen and Spiegelman (2001) J. Biol. Chem. 276:37731-34). GPAT3 mRNA is also significantly upregulated in the liver of ob/ob mice (FIG. 7B), a model of hepatic steatosis.

Along with a strong induction of microsomal GPAT activity during adipocyte differentiation, mitochondrial GPAT activity and mRNA levels are also elevated during differentiation (Yet et al., supra; Ericsson et al. (1997) J. Biol. Chem. 272:7298-305; Jerkins et al. (1995) J. Biol. Chem. 270:1416-21). However, those studies suggested that the contribution of N-ethylmaleimide-(NEM-) resistant mitochondrial GPAT to total GPAT activity in matured adipocytes was negligible (Yet et al., supra; Coleman et al. (1978), supra; Hajra et al. (2000) J. Biol. Chem. 275:9441-46). Here the inventors show that GPAT3 mRNA levels are induced >60-fold in differentiated adipocytes (FIG. 6A), concomitant with a significant increase in microsomal GPAT activity. Considering a pivotal role for PPARγ in adipogenesis, and the ability of PPARγ to induce GPAT3 transcription (FIG. 6D), the induction of GPAT3 might result from PPARγ activation during adipocyte differentiation. However, other PPARγ-independent mechanisms, such as the involvement of CCAAT/enhancer binding proteins (C/EBPs) and sterol regulatory element-binding proteins (SREBPs) are also possibly involved. In contrast to GPAT3 induction during adipocyte differentiation and PPARγ activation, GPAT3 mRNA is reduced in adipose tissue of ob/ob mice (FIG, 6B); this finding is consistent with the lipogenic role of GPAT3.

As such, the present invention provides GPAT3 antagonists, e.g., mouse and human GPAT3 inhibitory polynucleotides (i.e., polynucleotides that decrease GPAT3 levels and/or activity either directly or indirectly, e.g., antisense molecules, siRNAs, aptamers); GPAT3 inhibitory polypeptides (i.e., polypeptides that decrease GPAT3 levels and/or activity either directly or indirectly, e.g., fragments of GPAT3, such as soluble fragments containing the G3P or acyl-CoA interaction domains, and fusion proteins thereof); antagonistic anti-GPAT3 antibodies or antibody fragments (i.e., antibodies or antibody fragments that decrease GPAT3 activity and/or expression either directly or indirectly, including antagonistic antibodies and antibody fragments that bind full-length GPAT3 and/or GPAT3 fragments); and antagonistic small molecules (e.g., siRNAs, aptamers, and small inorganic and/or organic molecules or compounds), which may be used to suppress GPAT3-mediated acylation of G3P, and/or accumulation of TAG and/or TAG precursors (e.g., LPA, PA, and/or DAG), and consequently, which may be used in the diagnosis, prognosis, monitoring, treating, ameliorating and/or preventing disorders related to increased GPAT3 activity and/or disorders related to increased TAG levels and/or disorders treatable by decreasing GPAT3 activity or expression and/or TAG levels, i.e., GPAT3-associated conditions and/or conditions associated with TAG de novo synthesis. The present invention further provides GPAT3 agonists, e.g., GPAT3 polynucleotides and GPAT3 polypeptides (including full-length and/or fragments of GPAT3, such as a GPAT3 catalytic domain, and fusions thereof), agonistic anti-GPAT3 antibodies or antibody fragments (i.e., antibodies or antibody fragments that enhance GPAT3 activity and/or expression either directly or indirectly, including agonistic antibodies and antibody fragments that bind GPAT3 fragments), and agonist small molecules, which may be used to enhance GPAT3-mediated acylation of G3P, and/or accumulation of TAG and/or TAG precursors (e.g., LPA, PA, and/or DAG), and consequently, which may be used in the diagnosis, prognosis, monitoring, treating, ameliorating and/or preventing disorders related to decreased GPAT3 activity and/or disorders related to decreased TAG levels and/or disorders treatable by increasing GPAT3 activity or expression and/or TAG levels.

Disorders related to increased and decreased GPAT3 activities are described herein as “GPAT3-associated conditions” or “GPAT-2-associated disorders,” and include, without limitation, dyslipidemia (e.g., hyperlipidemia, hypertriglyceridemia, Type III hyperlipidemia), obesity, hypercholesterolemia, hepatic steatosis, cancer, skin disorders associated with altered lipid metabolism (e.g., acne vulgaris, dry skin), adiposity, type 2 diabetes (and complications associated therewith, such as dermopathy, retinopathy, neuropathy, and nephropathy), insulin resistance, hyperinsulinemia, hypertension, cardiovascular disease, atherosclerosis, arteriosclerosis, stroke, thrombosis, lipodystrophy (including congenital generalized lipodystrophy (Berardinelli-Seip syndrome), familial partial lipodystrophy (Dunnigan type, Kobberling type, and the mandibuloacral dysplasia type), and acquired forms of lipodystrophy such as acquired generalized lipodystrophy (Lawrence syndrome), acquired partial lipodystrophy (Barraquer-Simons syndrome), and lipodystrophy induced by antiviral treatments, e.g., treatment with HIV protease inhibitors), lipopenia, Reye's syndrome, Cushing's syndrome, metabolic syndrome (e.g., syndrome X), eating disorders (e.g., anorexia, bulimia), disorders and conditions related to skin homeostasis, disorders related to energy storage, nutrient absorption, and lipid metabolism, reduced or absent lactation, and low preterm birth weight (and complications thereof, such as defects in neural development);

The present invention further provides methods of screening for: 1) GPAT3 antagonists, e.g., mouse and human GPAT3 inhibitory polynucleotides (e.g., antisense, siRNA, aptamers); GPAT3 inhibitory polypeptides (e.g., G3P or acyl-CoA interacting fragments of GPAT3); antagonistic anti-GPAT3 antibodies and antibody fragments (including antibodies and antibody fragments that bind GPAT3 fragments); and antagonistic small molecules (e.g., siRNAs, aptamers, and small organic molecules or compounds); and 2) GPAT3 agonists, e.g., GPAT3 polynucleotides and polypeptides (including fragments of GPAT3, such as a GPAT3 catalytic domains) and fusions thereof; agonistic anti-GPAT3 antibodies and antibody fragments (including antibodies and antibody fragments that bind GPAT3 fragments); and agonistic small molecules. Such screening methods may be undertaken by, e.g., measuring changes in the level of expression of GPAT3 (e.g., levels of GPAT3 mRNA, cDNA, protein and/or protein fragments), or by measuring changes in the level of activity of GPAT3 (e.g., changes in levels of acylated GPAT3 product (e.g., LPA), changes in levels of nonacylated GPAT3 acceptor molecules (e.g., G3P), changes in levels of TAG and/or TAG precursors (e.g., LPA, PA, DAG), changes in the levels of CoA-SH byproducts, and/or changes in levels of acyl donors (e.g., lauroyl-CoA, oleoyl-CoA)).

The term “GPAT3” as used herein, where appropriate, refers to mammalian GPAT3, e.g., primate and/or rodent GPAT3, e.g., human and/or mouse GPAT3, and includes both GPAT3 polynucleotides (e.g., RNAs and DNAs, including the sequences disclosed herein, variants (e.g., analogs and homologs) and polymorphs thereof, and alleles of GPAT3) and GPAT3 polypeptides.

Accordingly, the present application provides GPAT3-related polynucleotides and polypeptides. The present invention also provides antibodies, i.e., intact antibodies and antigen-binding fragments thereof that bind to GPAT3, in particular, human and/or mouse GPAT3. In one embodiment, an anti-GPAT3 antibody inhibits or antagonizes at least one GPAT3-associated activity. For example, an anti-GPAT3 antibody may bind GPAT3 and interfere with (e.g., block, inhibit, neutralize) the interaction between GPAT3 and an acyl-CoA or the interaction between GPAT3 and G3P. An anti-GPAT3 antibody may also bind GPAT3 and interfere with GPAT3 enzymatic activity (e.g., acylation activity) by inducing, for example, a conformational change in GPAT3 amino acid tertiary and/or secondary structure. Alternatively, anti-GPAT3 antibodies may comprise agonistic antibodies that bind GPAT3 and enhance the interaction between GPAT3 and an acyl-CoA or the interaction between GPAT3 and G3P. An agonistic anti-GPAT3 antibody may also bind GPAT3 and stimulate GPAT3 enzymatic activity (e.g., acylation activity) by inducing, for example, a conformational change in GPAT3 amino acid tertiary and/or secondary structure. Thus, the antibodies of the invention may be used detect, and optionally inhibit (e.g., decrease, limit, block or otherwise reduce) or enhance (e.g., stimulate, increase, facilitate), a GPAT3 activity (e.g., interaction of GPAT3 with an acyl donor, interaction of GPAT3 with an acyl acceptor, GPAT3 catalytic activity, and/or modulation of TAG, MAG, LPA, PA, and/or G3P levels (e.g., accumulation or reduction in cell or tissue levels of TAG, MAG, LPA, PA, and/or G3P)). Thus, the anti-GPAT3 of the invention may be used to diagnose, prognose, monitor and/or treat or prevent disorders and conditions related to GPAT3 activity and/or disorders and conditions associated with synthesis (and/or accumulation) of TAG and/or TAG precursors.

GPAT3 Polynucleotides and Polypeptides

The present invention provides characterization of GPAT3, i.e., substrate affinity, cellular localization, enzymatic activity, and expression profiles. As such, the present invention relates to GPAT3 polynucleotides and polypeptides (e.g., full length and fragments of GPAT3 polynucleotides and polypeptides) and inhibitory GPAT3 polynucleotides and polypeptides (e.g., inhibitory full length and fragments of GPAT3 polynucleotides and polypeptides). The human GPAT3 (hGPAT3) nucleic acid sequence, which corresponds to GenBank Accession No. NM_032717, is set forth in SEQ ID NO:1. The human GPAT3 amino acid sequence is set forth in SEQ ID NO:2. The mouse GPAT3 (mGPAT3) nucleic acid sequence, which corresponds to GenBank Accession No. NM_{—}172715, is set forth in SEQ ID NO:3. The mouse GPAT3 amino acid sequence is set forth in SEQ ID NO:4. GPAT3 polypeptide refers to mammalian (e.g., human and mouse) GPAT3 proteins (including allelic variants) and fragments thereof, such as the amino acid sequences set forth in SEQ ID NO:2 and SEQ ID NO:4. GPAT3 polynucleotide refers to mammalian (e.g., human and mouse) GPAT3 nucleic acids (e.g., RNAs and DNAs (e.g., genomic DNA and cDNA), including the sequences disclosed herein, variants (e.g., analogs and homologs) and polymorphs thereof, and alleles of GPAT3) and fragments thereof, such as the nucleic acid sequences set forth in SEQ ID NO:1 and SEQ ID NO: 3.

The nucleic acids related to the present invention may comprise DNA or RNA and may be wholly or partially synthetic. Reference to a nucleotide sequence as set forth herein encompasses a DNA molecule with the specified sequence (or a complement thereof), and encompasses an RNA molecule with the specified sequence in which U is substituted for T, unless context requires otherwise.

The isolated polynucleotides related to the present invention may be used as hybridization probes and primers to identify and isolate nucleic acids having sequences identical to or similar to those encoding the disclosed polynucleotides. Hybridization methods for identifying and isolating nucleic acids include polymerase chain reaction (PCR), Southern hybridization, in situ hybridization and Northern hybridization, and are well known to those skilled in the art.

Hybridization reactions may be performed under conditions of different stringency. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1
Stringency Conditions

| Strin- | | | | |
|---|---|---|---|---|
| gency | Poly- | Hybrid | Hybridization | Wash |
| Condi- | nucleotide | Length | Temperature and | Temperature and |
| tion | Hybrid | (bp)¹ | Buffer² | Buffer² |
| A | DNA:DNA | >50 | 65° C.; 1xSSC -or- | 65° C.; 0.3xSSC |
| | | | 42° C.; 1xSSC, 50% | |
| | | | formamide | |
| B | DNA:DNA | <50 | T_{B}*; 1xSSC | T_{B}*; 1xSSC |
| C | DNA:RNA | >50 | 67° C.; 1xSSC -or- | 67° C.; 0.3xSSC |
| | | | 45° C.; 1xSSC, 50% | |
| | | | formamide | |
| D | DNA:RNA | <50 | T_{D}*; 1xSSC | T_{D}*; 1xSSC |
| E | RNA:RNA | >50 | 70° C.; 1xSSC -or- | 70° C.; 0.3xSSC |
| | | | 50° C.; 1xSSC, 50% | |
| | | | formamide | |
| F | RNA:RNA | <50 | T_{F}*; 1xSSC | T_{F}*; 1xSSC |
| G | DNA:DNA | >50 | 65° C.; 4xSSC -or- | 65° C.; 1xSSC |
| | | | 42° C.; 4xSSC, 50% | |
| | | | formamide | |
| H | DNA:DNA | <50 | T_{H}*; 4xSSC | T_{H}*; 4xSSC |
| I | DNA:RNA | >50 | 67° C.; 4xSSC -or- | 67° C.; 1xSSC |
| | | | 45° C.; 4xSSC, 50% | |
| | | | formamide | |
| J | DNA:RNA | <50 | T_{J}*; 4xSSC | T_{J}*; 4xSSC |
| K | RNA:RNA | >50 | 70° C.; 4xSSC -or- | 67° C.; 1xSSC |
| | | | 50° C.; 4xSSC, 50% | |
| | | | formamide | |
| L | RNA:RNA | <50 | T_{L}*; 2xSSC | T_{L}*; 2xSSC |
| M | DNA:DNA | >50 | 50° C.; 4xSSC -or- | 50° C.; 2xSSC |
| | | | 40° C.; 6xSSC, 50% | |
| | | | formamide | |
| N | DNA:DNA | <50 | T_{N}*; 6xSSC | T_{N}*; 6xSSC |
| O | DNA:RNA | >50 | 55° C.; 4xSSC -or- | 55° C.; 2xSSC |
| | | | 42° C.; 6xSSC, 50% | |
| | | | formamide | |
| P | DNA:RNA | <50 | T_{P}*; 6xSSC | T_{P}*; 6xSSC |
| Q | RNA:RNA | >50 | 60° C.; 4xSSC -or- | 60° C.; 2xSSC |
| | | | 45° C.; 6xSSC, 50% | |
| | | | formamide | |
| R | RNA:RNA | <50 | T_{R}*; 4xSSC | T_{R}*; 4xSSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the sequences of the polynucleotides and identifying the region or regions of optimal sequence complementarity. | | | | |
| ²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of | | | | |
| # A + T bases) + 4(# of G + C bases). For hybrids between 18 and 49 base pairs in length, Tₘ(° C.) = 81.5 + 16.6(log₁₀Na⁺) + 0.41(% G + C) − (600/N), where N is the number of bases in the hybrid, and Na⁺ is the concentration of sodium ions in the hybridization buffer (Na⁺ for 1xSSC = 0.165M). | | | | |

Additional examples of stringency conditions for polynucleotide hybridization are provided in Sambrook, J., E. F. Fritsch, and T. Maniatis, 1989, Molecular Cloning: A Laboratory Manual, Cold Spring Harbor Laboratory Press, Cold Spring Harbor, NY, chapters 9 and 11, and Current Protocols in Molecular Biology, 1995, F. M. Ausubel et al., eds., John Wiley & Sons, Inc., sections 2.10 and 6.3-6.4, incorporated herein by reference.

The isolated polynucleotides related to the present invention may be used as hybridization probes and primers to identify and isolate DNAs having sequences encoding allelic variants of the disclosed polynucleotides. Allelic variants are naturally occurring alternative forms of the disclosed polynucleotides that encode polypeptides that are identical to or have significant similarity to the polypeptides encoded by the disclosed polynucleotides. Preferably, allelic variants have at least 90% sequence identity (more preferably, at least 95% identity; most preferably, at least 99% identity) with the disclosed polynucleotides. Alternatively, significant similarity exists when the nucleic acid segments will hybridize under selective hybridization conditions (e.g., highly stringent hybridization conditions) to the disclosed polynucleotides.

The isolated polynucleotides related to the present invention may also be used as hybridization probes and primers to identify and isolate DNAs having sequences encoding polypeptides homologous to the disclosed polynucleotides. These homologs are polynucleotides and polypeptides isolated from a different species than that of the disclosed polypeptides and polynucleotides, or within the same species, but with significant sequence similarity to the disclosed polynucleotides and polypeptides. Preferably, polynucleotide homologs have at least 50% sequence identity (more preferably, at least 75% identity; most preferably, at least 90% identity) with the disclosed polynucleotides, whereas polypeptide homologs have at least 30% sequence identity (more preferably, at least 45% identity; most preferably, at least 60% identity) with the disclosed polypeptides. Preferably, homologs of the disclosed polynucleotides and polypeptides are those isolated from mammalian species.

Calculations of “homology” or “sequence identity” between two sequences may be performed by comparison methods well known in the art. For example, regarding identity, the sequences are aligned for optimal comparison purposes (e.g., gaps can be introduced in one or both of a first and a second amino acid or nucleic acid sequence for optimal alignment, and nonhomologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences, taking into account the number of gaps, and the length of each gap, which need to be introduced for optimal alignment of the two sequences.

The comparison of sequences and determination of percent sequence identity between two sequences may be accomplished using a mathematical algorithm. In a preferred embodiment, the percent identity between two amino acid sequences is determined using the Needleman and Wunsch ((1970) J. Mol. Biol. 48:444-53) algorithm, which has been incorporated into the GAP program in the GCG software package (available at www.gcg.com), using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package (available at www.gcg.com), using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine whether a molecule is within a sequence identity or homology limitation of the invention) is a Blossum 62 scoring matrix with a gap penalty of 12, a gap extend penalty of 4, and a frameshift gap penalty of 5. The percent identity between two amino acid or nucleotide sequences can also be determined using the algorithm of Meyers and Miller ((1989) CABIOS4:11-17), which has been incorporated into the ALIGN program (version 2.0), using a PAM120 weight residue table, a gap length penalty of 12 and a gap penalty of 4.

The isolated polynucleotides related to the present invention may also be used as hybridization probes and primers to identify cells and tissues that express the polypeptides related to the present invention and the conditions under which they are expressed.

Additionally, the function of the polypeptides related to the present invention may be directly examined by using the polynucleotides encoding the polypeptides to alter (i.e., enhance, reduce, or modify) the expression of the genes corresponding to the polynucleotides related to the present invention in a cell or organism. These “corresponding genes” are the genomic DNA sequences related to the present invention that are transcribed to produce the mRNAs from which the polynucleotides related to the present invention are derived.

Altered expression of the genes related to the present invention may be achieved in a cell or organism through the use of various inhibitory polynucleotides, such as antisense polynucleotides, siRNAs, and ribozymes that bind and/or cleave the mRNA transcribed from the genes related to the invention (see, e.g., Galderisi et al. (1999) J. Cell Physiol. 181:251-57; Sioud (2001) Curr. Mol. Med. 1:575-88). Inhibitory polynucleotides to GPAT3 may be useful as TAG, MAG, LPA and/or PA antagonists and, as such, may also be useful in preventing or treating disorders related to TAG, MAG, LPA and/or PA synthesis and/or accumulation. Inhibitory polynucleotides may also consist of aptamers, i.e., polynucleotides that bind to and regulate protein activity, e.g., the activity of human GPAT3. Aptamers are described in the literature, see, e.g., Nimjee et al. (2005) Annu. Rev. Med 56:555-83; Patel (1997) Curr. Opin. Chem. Biol. 1:32-46.

The inhibitory polynucleotides of the present invention also include triplex-forming oligonucleotides (TFOs) that bind in the major groove of duplex DNA with high specificity and affinity (Knauert and Glazer (2001) Hum. Mol. Genet. 10:2243-51). Expression of the genes related to the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides of the present invention are short interfering RNA (siRNA) molecules (preferably 19-25 nucleotides; most preferably 19 or 21 nucleotides) useful for RNA interference (RNAi) (e.g., Bass (2001) Nature 411:428-29). The siRNA molecules of the present invention may be generated by a variety of methods that are well known in the art (Fire et al., U.S. Pat. No. 6,506,559; Yu et al. (2002) Proc. Natl. Acad. Sci. USA 99:6047-52; Elbashir et al. (2001) Nature 411:494-98; Yu et al., supra; Sui et al. (2002) Proc. Natl. Acad. Sci. USA 99:5515-20; Paddison et al. (2002) Proc. Natl. Acad Sci. USA 99:1443-48; Arts et al. (2003) Genome Res. 13:2325-32). The siRNA molecules targeted to the polynucleotides related to the present invention can be designed based on criteria well known in the art (e.g., Elbashir et al. (2001) EMBO J. 20:6877-88; Reynolds et al. (2004) Nature Biotechnol. 22:326-30).

In some embodiments of the invention, the inhibitory polynucleotide, e.g., siRNA molecule or antisense molecule, targets exon 3 of GPAT3 (e.g., the nucleic acid sequence encoding about amino acids 59-116 of mouse GPAT3).

Altered expression of the genes related to the present invention in an organism may also be achieved through the creation of nonhuman transgenic animals into whose genomes polynucleotides related to the present invention have been introduced. Such transgenic animals include animals that have multiple copies of a gene (i.e., the transgene) of the present invention. A tissue-specific regulatory sequence(s) may be operably linked to the transgene to direct expression of a polypeptide related to the present invention to particular cells or a particular developmental stage. Methods for generating transgenic animals via embryo manipulation and microinjection, particularly animals such as mice, have become conventional and are well known in the art (e.g., Bockamp et al. (2002) Physiol. Genomics 11:115-32).

Altered expression of the genes related to the present invention in an organism may also be achieved through the creation of animals whose endogenous genes corresponding to the polynucleotides related to the present invention have been disrupted through insertion of extraneous polynucleotide sequences (i.e., a knockout animal). The coding region of the endogenous gene may be disrupted, thereby generating a nonfunctional protein. Alternatively, the upstream regulatory region of the endogenous gene may be disrupted or replaced with different regulatory elements, resulting in the altered expression of the still-functional protein. Methods for generating knockout animals include homologous recombination and are well known in the art (e.g., Wolfer et al. (2002) Trends Neurosci. 25:336-40).

The isolated polynucleotides of the present invention also may be operably linked to an expression control sequence and/or ligated into an expression vector for recombinant production of the polypeptides (including active fragments and/or fusion polypeptides thereof) related to the present invention. An expression vector, as used herein, is intended to refer to a nucleic acid molecule capable of transporting another nucleic acid to which it has been linked and includes plasmids, yeast artificial chromosomes, viral vectors, etc. In the present specification, plasmid and vector may be used interchangeably, as the plasmid is the most commonly used form of vector. In general, expression vectors of utility in recombinant DNA techniques are often in the form of plasmids.

Suitable vectors can be chosen or constructed, containing appropriate regulatory sequences, including promoter sequences, terminator sequences, polyadenylation sequences, enhancer sequences, selectable marker genes and other sequences, e.g., sequences that regulate replication of the vector in the host cells (e.g., origins of replication), as appropriate. For further details see, for example, Molecular Cloning: a Laboratory Manual: 2nd ed., Sambrook et al., Cold Spring Harbor Laboratory Press, 1989 and Current Protocols in Molecular Biology, 2nd ed., Ausubel et al. (eds.) John Wiley & Sons, 1992.

In one embodiment, the polynucleotides related to the present invention are used to create recombinant GPAT3 agonists and antagonists. Exemplary GPAT3 agonists include, but are not limited to, wild type GPAT3 (polypeptide or polynucleotide) and active (e.g., enzymatically active) fragments thereof. Such agonists may be useful in regulating TAG biosynthesis, and consequently, in the treatment of lipodystrophy and other disorders in which it is desirable to enhance TAG synthesis and/or levels of PA, LPA and/or DAG. In another embodiment, the polynucleotides related to the present invention are used to create GPAT3 antagonists, e.g., GPAT3 inhibitory polynucleotides; soluble GPAT3 polypeptides (including fragments (e.g., acyl-CoA- and/or G3P-interacting fragments) and/or fusion proteins thereof); antagonistic anti-GPAT3 antibodies; and/or antagonistic small molecules; etc. Such antagonists may be useful in regulating TAG biosynthesis, and consequently, in the treatment of obesity, type 2 diabetes, and other disorders where it is desirable to decrease TAG synthesis and/or levels of PA, LPA and/or DAG, or to increase cellular stores of G3P.

Methods of creating fusion polypeptides, i.e., a first polypeptide moiety linked with a second polypeptide moiety, are well known in the art. For example, a GPAT3 polypeptide may be fused directly or indirectly through a “linker” sequence (e.g., a peptide linker of about 2 to 20, more preferably less than 10, amino acids in length) to a second polypeptide moiety, e.g., an immunoglobulin or a fragment thereof (e.g., an Fc binding fragment thereof), a heterologous sequence (e.g., sequences encoding glutathione-S-transferase (GST), Lex A, thioredoxin (TRX) or maltose-binding protein (MBP); signal sequences; and tag sequences), or a homologous sequence (e.g., a domain from another GPAT3 polynucleotide). The second polypeptide moiety is preferably soluble. In some embodiments, the second polypeptide moiety enhances the half-life, (e.g., the serum half-life) of the linked polypeptide. In preferred embodiments, the second polypeptide includes at least a region of an immunoglobulin polypeptide. Immunoglobulin fusion polypeptides are known in the art and are described in, e.g., U.S. Pat. Nos. 5,516,964; 5,225,538; 5,428,130; 5,514,582; 5,714,147; and 5,455,165, all of which are hereby incorporated by reference in their entireties.

A fusion protein of the invention may be produced by standard recombinant DNA techniques such as cloning and subcloning, chemical synthesis, and PCR (see, for example, Current Protocols in Molecular Biology, Ausubel et al. (eds.), John Wiley & Sons, 1992). Moreover, many expression vectors are commercially available that encode a fusion moiety (e.g., an Fc region of an immunoglobulin heavy chain). A GPAT3-encoding nucleic acid may be cloned into such an expression vector such that the fusion moiety is linked in-frame to the immunoglobulin protein.

A further aspect of the present invention provides a host cell comprising a nucleic acid as disclosed herein. A still further aspect provides a method comprising introducing such a nucleic acid into a host cell. The introduction may employ any available technique, including calcium phosphate transfection, DEAE-Dextran, electroporation, gene-gun transfer, liposome-mediated transfection, transduction using retrovirus or other viruses, baculovirus infection, calcium chloride transfection or transformation, and transfection using bacteriophage. The introduction may be followed by causing or allowing expression from the nucleic acid, e.g., by culturing host cells under conditions for expression of the gene. Such techniques are well known in the art.

A number of cell lines and primary cells may act as suitable host cells for recombinant expression of the polypeptides related to the present invention. Host cells include mammalian cells (e.g., COS cells, CHO cells, 293 cells, primary explants, etc.), lower eukaryotic cells (e.g., yeast cells), insect cells (e.g., using baculovirus/Sf9 expression systems), and prokaryotic cells (e.g., E. coli). If the polypeptides related to the present invention are made in yeast or bacteria, it may be necessary to modify them by, for example, phosphorylation or glycosylation of appropriate sites, or by refolding the recombinant protein in order to obtain functionality. Such covalent modifications may be accomplished using well-known chemical or enzymatic methods, and general methods of refolding are disclosed in, e.g., Kohno (1990) Meth. Enzymol. 185:187-95. Other appropriate methods are disclosed in, e.g., EP 0433225 and U.S. Pat. No. 5,399,677.

Following recombinant expression in the appropriate host cells, the recombinant polypeptides of the present invention may be purified from cell extracts using known purification processes, such as immunoprecipitation, gel filtration, affinity chromatography, and ion exchange (anion or cation as appropriate) chromatography. Preferably, the isolated recombinant protein is purified so that it is substantially free of other mammalian proteins. Additionally, various purification processes may also be used to purify the polypeptides of the present invention from other sources, including natural sources (e.g., from the milk of transgenic animals). Alternatively, the polypeptides may also be recombinantly expressed in a form that facilitates purification (e.g., fusions containing GST or MPB, or fusions containing epitope tags, e.g., myc or FLAG tags). Kits for expression and purification of such fusion proteins are commercially available from, e.g., New England BioLabs (Beverly, Mass.), Pharmacia (Piscataway, N.J.), and Invitrogen.

The polypeptides related to the present invention, including GPAT3 agonists and antagonists, may also be produced by known conventional chemical synthesis. Methods for chemically synthesizing such polypeptides are well known to those skilled in the art. Such chemically synthetic polypeptides may possess biological properties in common with the natural, purified polypeptides, and thus may be employed as biologically active or immunological substitutes for the natural polypeptides.

The polypeptides related to the present invention, including GPAT3 agonists and antagonists, also encompass molecules that are structurally different from the disclosed polypeptides (e.g., which have a slightly altered sequence), but have substantially the same biochemical properties as the disclosed polypeptides (e.g., are changed only in functionally nonessential amino acid residues). Such molecules include naturally occurring allelic variants and deliberately engineered variants containing alterations, substitutions, replacements, insertions, or deletions. Techniques for such alterations, substitutions, replacements, insertions, or deletions are well known to those skilled in the art. In some embodiments, the polypeptide moiety is provided as a variant polypeptide having mutations in the naturally occurring sequence (wild type) that results in a sequence more resistant to proteolysis (relative to the nonmutated sequence).

GPAT3 polypeptides, fragments and/or fusion polypeptides thereof, and recombinant and/or natural forms thereof, may be used to screen for agents (e.g., other GPAT3 agonists or antagonists, e.g., anti-GPAT3 antibodies) that are capable of binding GPAT3 and/or regulating GPAT3 activity, as described further herein. Binding assays utilizing a desired binding protein, immobilized or not, are well known in the art and may be used for this purpose with the polypeptides related to the present invention, including the GPAT3 antagonists and agonists of the invention, e.g., GPAT3 polynucleotides and polypeptides. Purified cell-based or protein-based (cell-free) screening assays may be used to identify such agents. For example, GPAT3 polypeptides may be immobilized in purified form on a carrier and binding of potential ligands to purified GPAT3 may be measured.

Antibodies

In other embodiments, the invention provides GPAT3 agonists and antagonists as antibodies, i.e., intact antibodies and antigen binding fragments thereof, that specifically bind to GPAT3 and/or fragments of GPAT3, preferably mammalian (e.g., human or mouse) GPAT3. In one embodiment, the antibodies are inhibitory antibodies, i.e., they inhibit at least one GPAT3 activity (e.g., accumulation of TAG) and may be useful in diagnosing, prognosing, monitoring and/or treating disorders related to TAG dysregulation. Additionally, the invention provides agonistic antibodies, i.e., antibodies that enhance at least one GPAT3 activity (e.g., accumulation of TAG) and may be useful in diagnosing, prognosing, monitoring and/or treating disorders related to TAG dysregulation. Additionally, the invention provides anti-GPAT3 antibodies that specifically bind to GPAT3, but do not inhibit or increase GPAT3 activity (i.e., detecting antibodies); such antibodies may be used to detect the presence of, e.g., GPAT3 protein, e.g., as part of a kit for diagnosing, prognosing, and/or monitoring a disorder(s) related to GPAT3 activity. In one embodiment, the antibody is directed to GPAT3, preferably mammalian GPAT3, more preferably human GPAT3. In another embodiment, the antibody is a monoclonal or single specificity antibody. The antibodies may also be human, humanized, chimeric, or in vitro-generated antibodies against human or mouse GPAT3.

One of skill in the art will recognize that, as used herein, the term “antibody” refers to a protein comprising at least one, and preferably two, heavy (H) chain variable regions (abbreviated herein as VH), and at least one and preferably two light (L) chain variable regions (abbreviated herein as VL). The antibody may further include a heavy and light chain constant region to thereby form a heavy and light immunoglobulin chain, respectively. In one embodiment, the antibody is a tetramer of two heavy immunoglobulin chains and two light immunoglobulin chains, wherein the heavy and light immunoglobulin chains are interconnected, e.g., by disulfide bonds.

The antigen binding fragment of an antibody (or simply “antibody portion,” or “fragment”), as used herein, refers to one or more fragments of a full-length antibody that retain the ability to specifically bind to an antigen (e.g., CD3). Examples of binding fragments encompassed within the term “antigen binding fragment” of an antibody include, but are not limited to: (i) an Fab fragment, a monovalent fragment consisting of the VL, VH, CL and CH1 domains; (ii) an F(ab′)₂ fragment, a bivalent fragment comprising two Fab fragments linked by a disulfide bridge at the hinge region; (iii) an Fd fragment consisting of the VH and CHI domains; (iv) an Fv fragment consisting of the VL and VH domains of a single arm of an antibody; (v) a dAb fragment, which consists of a VH domain; and (vi) an isolated complementarity determining region (CDR). Furthermore, although the two domains of the Fv fragment, VL and VH, are encoded by separate genes, they may be joined, using recombinant methods, by a synthetic linker that enables their production as a single protein chain in which the VL and VH regions pair to form monovalent molecules (known as single chain Fv (scFv)). Such single chain antibodies are also encompassed within the term “antigen binding fragment” of an antibody. These antibody fragments are obtained using conventional techniques known to those skilled in the art, and the fragments are screened for utility in the same manner as are intact antibodies.

Antibody molecules to the polypeptides of the present invention, e.g., antibodies to GPAT3, may be produced by methods well known to those skilled in the art. GPAT3 proteins of the invention may also be used to immunize animals to obtain polyclonal and monoclonal antibodies that react with the GPAT3 protein and which may inhibit or enhance the interaction of acyl-CoA and/or G3P with GPAT3, or which may inhibit or enhance GPAT3 catalytic activity. A full-length polypeptide of the present invention may be used as the immunogen, or, alternatively, antigenic peptide fragments of the polypeptides may be used. An antigenic peptide of a polypeptide of the present invention comprises at least seven continuous amino acid residues and encompasses an epitope such that an antibody raised against the peptide forms a specific immune complex with the polypeptide. Preferably, the antigenic peptide comprises at least 10 amino acid residues, more preferably at least 15 amino acid residues, even more preferably at least 20 amino acid residues, and most preferably at least 30 amino acid residues.

In a further improvement to this procedure, a method for identifying a clinically relevant epitope on an immunogen, and a correlative method for selecting an antibody that binds immunospecifically to the relevant epitope with high affinity, are disclosed in, e.g., PCT international patent publication WO 99/53049, which is hereby incorporated by reference herein in its entirety. Exemplary epitopes generally useful for targeting lipid acyltransferases, e.g., G3P interaction domains and catalytic domains, are discussed in, e.g., Coleman and Lee (2004), supra.

Monoclonal antibodies may be produced by generation of hybridomas in accordance with known methods, or by screening a recombinant combinatorial immunoglobulin library (e.g., an antibody phage display library) with a polypeptide related to the present invention (e.g., mouse and human GPAT3 and fragments thereof) to thereby isolate immunoglobulin library members that bind to the polypeptides related to the present invention. The “combinatorial antibody display” method is well known and was developed to identify and isolate antibody fragments having a particular antigen specificity, and may be utilized to produce monoclonal antibodies.

Polyclonal sera and antibodies may be produced by immunizing a suitable subject with a polypeptide of the present invention. The antibody titer in the immunized subject may be monitored over time, and the antibody molecules directed against a polypeptide of the present invention may be isolated from the subject or culture media and further purified by well-known techniques.

Fragments of antibodies to the polypeptides of the present invention may be produced by cleavage of the antibodies in accordance with methods well known in the art. For example, immunologically active Fab and F(ab′)₂ fragments may be generated by treating the antibodies with an enzyme such as pepsin.

Additionally, chimeric, humanized, and single-chain antibodies to the polypeptides of the present invention, comprising both human and nonhuman portions, may be produced using standard recombinant DNA techniques and/or a recombinant combinatorial immunoglobulin library. The production of chimeric, humanized, and single-chain antibodies is well known in the art (see, e.g., Morrison (1985) Science 229:1202-07; Oi et al. (1986) BioTechniques 4:214-21; Queen et al., U.S. Pat. Nos. 5,585,089; 5,693,761; 5,693,762, the contents of all of which are hereby incorporated by reference herein). Humanized or CDR-grafted antibody molecules or immunoglobulins may be produced by standard procedures (see, e.g., U.S. Pat. No. 5,225,539; Jones et al. (1986) Nature 321:552-25; Verhoeyan et al. (1988) Science 239:1534; Beidler et al. (1988) J. Immunol. 141:4053-60; Winter, U.S. Pat. No. 5,225,539, the contents of all of which are hereby incorporated by reference herein). Human antibodies may be produced using transgenic nonhuman animals that are modified so as to produce fully human antibodies rather than the animal's endogenous antibodies in response to challenge by an antigen (see PCT international patent publication WO 94/02602, WO 96/33735 and WO 96/34096). Monoclonal, chimeric, human and humanized antibodies that have been modified by, e.g., deleting, adding, or substituting other portions of the antibody, e.g., the constant region, are also within the scope of the invention. As nonlimiting examples, an antibody can be modified by deleting the constant region, by replacing the constant region with another constant region, e.g., a constant region meant to increase half-life, stability, or affinity of the antibody, or a constant region from another species or antibody class, and by modifying one or more amino acids in the constant region to alter, for example, the number of glycosylation sites, effector cell function, Fc receptor (FcR) binding, complement fixation, etc.

Antibodies with altered function, e.g., altered affinity for an effector ligand, such as FcR on a cell, or the C1 component of complement, can be produced by replacing at least one amino acid residue in the constant portion of the antibody with a different residue (see, e.g., EP 388,151, U.S. Pat. Nos. 5,624,821 and 5,648,260, the contents of which are hereby incorporated by reference herein in their entireties).

In addition to antibodies for use in the instant invention, other molecules may also be employed to modulate the activity of GPAT3. Such molecules include small modular immunopharmaceutical (SMIP™) drugs (Trubion Pharmaceuticals, Seattle, Wash.). SMIPs are single-chain polypeptides composed of a binding domain for a cognate structure such as an antigen, a counter receptor or the like, a hinge-region polypeptide having either one or no cysteine residues, and immunoglobulin CH2 and CH3 domains (see also www.trubion.com). SMIPs and their uses and applications are disclosed in, e.g., U.S. Published Patent Appln. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

Anti-GPAT3 antibodies of the invention may be useful for isolating, purifying, and/or detecting GPAT3 polypeptides and GPAT3 polypeptide fragments (or fusions thereof), in supernatants, cellular lysates, or on the cell surface. Antibodies disclosed in the invention may be also used diagnostically to monitor, e.g., GPAT3 polypeptide levels, as part of a clinical testing procedure, or clinically to target a therapeutic modulator to a cell or tissue comprising the antigen of the antibody. For example, a therapeutic, such as a small molecule or other therapeutic of the invention, may be linked to an anti-GPAT3 antibody in order to target the therapeutic to the cell or tissue expressing GPAT3. Antagonistic and agonistic antibodies (preferably monoclonal antibodies) that bind to GPAT3 polypeptides may also be useful in the treatment of a disease(s) related to GPAT3 activity, and/or a GPAT3-associated condition(s). Thus, the present invention further provides compositions comprising an inhibitory (antagonistic) antibody that specifically binds to GPAT3 and decreases, limits, blocks, or otherwise reduces GPAT3 activity. The present invention further provides compositions comprising a stimulatory (agonistic) antibody that specifically binds to GPAT3 and increases or otherwise enhances GPAT3 activity. Similarly, anti-GPAT3 antibodies may be useful in isolating, purifying, detecting, and/or diagnostically monitoring GPAT3, and/or clinically targeting a therapeutic modulator to a cell or tissue comprising GPAT3.

Screening Assays

The GPAT3 polynucleotides and polypeptides may be used in screening assays to identify pharmacological agents or lead compounds for agents that are capable of modulating the activity of GPAT3 in a cell or organism and are thereby potential regulators of TAG synthesis and disorders associated with TAG dysregulation. For example, samples containing GPAT3 may be contacted with one of a plurality of test compounds (either biological agents or small organic molecules), and the activity of GPAT3 in each of the treated samples can be compared with the activity of GPAT3 in untreated samples or in samples contacted with different test compounds. Such comparisons will determine whether any of the test compounds results in: 1) a substantially decreased level of expression or activity of GPAT3, thereby indicating an antagonist of GPAT3, or 2) a substantially increased level of expression or activity of GPAT3, thereby indicating an agonist of GPAT3. In one embodiment, the identification of test compounds capable of modulating GPAT3 activity is performed using high-throughput screening assays, such as BIACORE® (Biacore International AB, Uppsala, Sweden), BRET (bioluminescence resonance energy transfer), and/or FRET (fluorescence resonance energy transfer) assays, as well as ELISA and/or cell-based assays.

As GPATs increases levels of TAG, screens for agonists or antagonists of GPAT3 activity may employ well-established methods for analyzing lipid biosynthesis, or may follow the protocols described in the Examples. Thus, one may contact a cell or sample containing GPAT3 with a test compound, and determine if the test compound modulates GPAT3 expression by, e.g., Western or Northern Analysis, PCR, immunohistochemistry, in situ hybridization, differential display, etc. Alternatively, one may contact a cell or sample containing GPAT3 with a test compound and determine if the test compound modulates GPAT3 activity. GPAT3 activity may be measured by a variety of methods, including measuring changes in levels of acylated product (e.g., LPA), changes in levels of nonacylated acceptor molecules (e.g., G3P), changes in levels of TAG and/or TAG precursors (e.g., LPA, PA, DAG), changes in levels of CoA-SH byproduct, and changes in levels of acyl donors (e.g., lauroyl-CoA, oleoyl-CoA). As shown in the Examples, using, e.g., thin layer chromatography or butanol extraction, one may employ a [¹⁴C]-labeled acceptor (G3P) or various donor molecules (e.g., lauroyl-CoA, palmitoyl-CoA, oleoyl-CoA) in a method of measuring GPAT3 activity. Other acyl-CoA donors and useful labels (e.g., ³H) are well known in the art, and additional methods for acyltransferase activity are disclosed throughout the literature (see, e.g., Coleman and Lee, supra; Chen and Farese (2000), supra; Chen and Farese (2005), supra; Yamazaki et al. (2005) J. Biol. Chem. 280:21506-14; Coleman (1992) Meth. Enzymol. 209:98-104; and U.S Patent Appln. 2002/0127627 A1).

Small Molecules

Decreasing GPAT3 activity in an organism (or subject) afflicted with (or at risk for) a disorder related to enhanced GPAT3 expression and/or activity or a disorder related to increased TAG levels or TAG accumulation, e.g., obesity, type 2 diabetes, etc., or in a cell from such an organism or subject, may also be achieved through the use of small molecules (usually organic small molecules) that antagonize, i.e., inhibit the activity of, GPAT3. Novel antagonistic small molecules may be identified by the screening methods described herein and may be used in the treatment, amelioration and/or prevention methods of the present invention described herein.

Conversely, increasing GPAT3 activity in an organism (or subject) afflicted with (or at risk for) a disorder related to decreased GPAT3 expression and/or activity or a disorder related to decreased TAG levels, e.g., lipodystrophy, may also be achieved through the use of small molecules (usually organic small molecules) that agonize, i.e., enhance the activity of, GPAT3. Novel agonistic small molecules may be identified by the screening methods described herein and may be used in the treatment, amelioration and/or prevention methods of the present invention described herein.

The term small molecule refers to compounds that are not macromolecules (see, e.g., Karp (2000) Bioinformatics Ontology 16:269-85; Verkman (2004) AJP-Cell Physiol. 286:465-74). Thus, small molecules are often considered those compounds that are, e.g., less than one thousand daltons (e.g., Voet and Voet, Biochemistry, 2^{nd} ed., ed. N. Rose, Wiley and Sons, New York, 14 (1995)). For example, Davis et al. ((2005) Proc. Natl. Acad. Sci. USA 102:5981-86) use the phrase small molecule to indicate folates, methotrexate, and neuropeptides, whereas Halpin and Harbury ((2004) PLos Biology 2:1022-30) use the phrase to indicate small molecule gene products, e.g., DNAs, RNAs and peptides. Examples of natural small molecules include, but are not limited to, cholesterols, neurotransmitters, aptamers, and siRNAs; synthesized small molecules include, but are not limited to, various chemicals listed in numerous commercially available small molecule databases, e.g., FCD (Fine Chemicals Database), SMID (Small Molecule Interaction Database), ChEBI (Chemical Entities of Biological Interest), and CSD (Cambridge Structural Database) (see, e.g., Alfarano et al. (2005) Nuc. Acids Res. Database Issue 33:D416-24).

Methods for Diagnosing, Prognosing, and Monitoring the Progress of Disorders and Conditions Related to GPAT3 Activity

The present invention provides methods for diagnosing, prognosing, and monitoring the progress of disorders and conditions related to GPAT3 in a subject (e.g., conditions that directly or indirectly involve increases or decreases in the activity of GPAT3) by detecting, e.g., an upregulation or a downregulation of GPAT3 activity, e.g., by detecting the upregulation of human GPAT3, including but not limited to the use of such methods in human subjects. These methods may be performed by utilizing prepackaged diagnostic kits comprising at least one of the group comprising a GPAT3 polynucleotide or fragments thereof, a GPAT3 polypeptide or fragments thereof (including fusion proteins thereof), antibodies to a GPAT3 polypeptide or derivatives thereof, or modulators of GPAT3 polynucleotides and/or polypeptides as described herein, which may be conveniently used, for example, in a clinical setting. A skilled artisan will recognize that other indirect methods may be used to confirm, e.g., the upregulation of GPAT3, e.g., human GPAT3, such as measuring changes in the mass of adipose tissue.

“Diagnostic” or “diagnosing” means identifying the presence or absence of a pathologic condition. Diagnostic methods include detecting regulation of the level of expression of GPAT3 and/or the level of activity GPAT3 by determining a test amount of the level of expression of GPAT3 (e.g., level of mRNA, cDNA, and/or polypeptide, including fragments thereof) and/or level of activity of GPAT3 (e.g., level of acyl transferase activity, level of conversion of G3P to LPA, accumulation of LPA, PA, DAG and/or TAG, reduction in G3P levels, reduction in acyl-CoA levels, etc.) in a biological sample from a subject (human or nonhuman mammal), and comparing the test amount with a normal amount or range (e.g., a reference amount, such an amount or range from an individual(s) known not to suffer from disorders related to GPAT3 activity). Although a particular diagnostic method may not provide a definitive diagnosis of disorders related GPAT3 activity, it suffices if the method provides a positive or negative indication that aids in diagnosis.

The present invention also provides methods for prognosing such disorders by detecting changes in the level (increases or decreases) of GPAT3 expression or activity. “Prognostic” or “prognosing” means predicting the probable development and/or severity of a pathologic condition. Prognostic methods include determining the test amount of a gene product of GPAT3 and/or the level of activity of GPAT3 contained in a biological sample from a subject, and comparing the test amount or activity level to a prognostic amount or range (i.e., an amount or range from individuals with varying severities of disorders related to GPAT3 activity and/or disorders associated with TAG dysregulation) of the gene product and/or level of activity of GPAT3. Various amounts of the GPAT3 gene product or level of activity of GPAT3 in a test sample are consistent with certain prognoses for disorders related to GPAT3 activity and/or disorders associated with TAG dysregulation. The detection of an amount of GPAT3 gene product or GPAT3 level of activity, e.g., at a particular prognostic level, provides a prognosis for the subject.

The present invention also provides methods for monitoring the progress or course of such disorders or the course of treatment of disorders related to GPAT3 activity (and/or disorders associated with TAG dysregulation) by detecting, e.g., the upregulation or downregulation of GPAT3 activity or expression. Monitoring methods include determining the test amounts of a gene product of GPAT3 and/or level of activity of GPAT3 in biological samples taken from a subject at a first and second time, and comparing the amounts. A change in amount of a GPAT3 gene product between the first and second times indicates a change in the course of GPAT3-related conditions or disorders. Such monitoring assays are also useful for evaluating the efficacy of a particular therapeutic intervention in patients being treated for GPAT3-associated conditions and/or conditions resulting in TAG dysregulation, e.g., measuring and comparing the levels of GPAT3 activity or expression before and after administration of a therapeutic treatment.

Increased GPAT3 activity in the methods outlined above may be detected in a variety of biological samples, including bodily fluids (e.g., whole blood, plasma, and urine), cells (e.g., whole cells, cell fractions, and cell extracts), and other tissues. Biological samples also include sections of tissue, such as biopsies and frozen sections taken for histological purposes. Preferred biological samples include adipose, heart, liver, kidney, muscle, thyroid, testis, and intestine. It will be appreciated that analysis of a biological sample need not necessarily require removal of cells or tissue from the subject. For example, appropriately labeled agents that bind GPAT3 gene products (e.g., antibodies, nucleic acids) can be administered to a subject and visualized (when bound to the target) using standard imaging technology (e.g., CAT, NMR (MRI), and PET).

In the diagnostic and prognostic assays of the present invention, the GPAT3 gene product is detected and quantified to yield a test amount. The test amount is then compared with a normal amount or range. Particular methods of detection and quantitation of GPAT3 gene products are described below.

Normal amounts or baseline levels of GPAT3 gene products may be determined for any particular sample type and population. Generally, baseline (normal) levels of GPAT3 protein or mRNA are determined by measuring respective amounts of GPAT3 protein or mRNA in a biological sample from normal (i.e., healthy) subjects. Alternatively, normal values of GPAT3 gene product(s) may be determined by measuring the amount in healthy cells or tissues taken from the same subject from which the diseased (or possibly diseased) test cells or tissues were taken. The amount of GPAT3 gene product(s) (either the normal amount or the test amount) may be determined or expressed on a per cell, per total protein, or per volume basis. To determine the baseline amount of a sample, one can measure the level of a constitutively expressed gene product or other gene product expressed at known levels in cells of the type from which the biological sample was taken.

It will be appreciated that the assay methods of the present invention do not necessarily require measurement of absolute values of GPAT3 gene products because relative values are sufficient for many applications of these methods. It will also be appreciated that in addition to the quantity or abundance of GPAT3 gene products, variant or abnormal GPAT3 gene products or their expression patterns (e.g., mutated transcripts, truncated polypeptides) may be identified by comparison to normal gene products and expression patterns.

Whether the expression of a particular gene in two samples is significantly similar or significantly different, e.g., significantly above or significantly below a given level, depends on the gene itself and, inter alia, its variability in expression between different individuals or different samples. It is within the skill of those in the art to determine whether expression levels are significantly similar or different. Factors such as genetic variation, e.g., in GPAT3 expression levels, between individuals, species, organs, tissues, or cells may be taken into consideration (if necessary) when determining whether the level of expression, e.g., of human GPAT3, between two samples is significantly similar or significantly different, e.g., significantly above or below a given level. As a result of the natural heterogeneity in gene expression between individuals, species, organs, tissues, or cells, phrases such as “significantly similar,” “significantly greater,” “significantly lower,” “significantly above” and the like cannot be defined as a precise percentage or value, but rather can be ascertained by one skilled in the art upon practicing the invention.

Uses of Molecules Related to GPAT3 Activity in Therapy

The inventors have demonstrated, inter alia, the following: 1) overexpression of mouse or human GPAT3 in mammalian and insect cells results in increased acylation of G3P; 2) the acyl transferase activity of GPAT3 is specific for G3P as an acyl acceptor; 3) GPAT3 is an NEM-sensitive acyl transferase; 4) GPAT3 expression results in the formation of neutral rather than polar lipids; 5) GPAT3 localizes to the endoplasmic reticulum rather than the mitochondria; 6) mouse and human GPAT3 are expressed in metabolically active tissues (e.g., heart, adipose, kidney, intestine); and 7) GPAT3 expression is actively upregulated during adipocyte differentiation, decreased in adipose tissue of ob/ob mice, and upregulated by PPARY activation. The above results indicate that the disclosed methods for using molecules related to GPAT3 activity, e.g., agonists and antagonists of GPAT3, to treat GPAT3-associated conditions and disorders, will be particularly useful for treating such disorders in humans.

The GPAT3-related molecules disclosed herein, including modulators of mammalian, e.g., mouse and human GPAT3 polynucleotide and/or polypeptide activity identified using the methods described herein, may be used in vitro, ex vivo, or incorporated into pharmaceutical compositions and administered to individuals (e.g., human subjects) in vivo to treat, ameliorate, or prevent, e.g., disorders related to GPAT3 activity and disorders related to TAG synthesis and/or accumulation, by administration of a GPAT3 antagonist (e.g., GPAT3 inhibitory polynucleotides (i.e., polynucleotides that decrease GPAT3 levels and/or activity either directly or indirectly, e.g., antisense, siRNA, aptamers); GPAT3 inhibitory polypeptides (i.e., polypeptides that decrease GPAT3 levels and/or activity either directly or indirectly, e.g., fragments of GPAT3, such as soluble fragments containing the G3P or acyl-CoA interaction domains, and fusion proteins thereof); antagonist anti-GPAT3 antibodies or antibody fragments (i.e., antibodies or antibody fragments that decrease GPAT3 activity and/or expression either directly or indirectly, including antibodies and antibody fragments that bind GPAT3 fragments); and antagonistic small molecules (e.g., siRNAs, aptamers, and small organic molecules or compounds)), or a GPAT3 agonist (e.g., GPAT3 polynucleotides and GPAT3 polypeptides (including full-length and/or fragments of GPAT3, such as a GPAT3 catalytic domain, and fusions thereof); agonistic anti-GPAT3 antibodies or antibody fragments (i.e., antibodies or antibody fragments that enhance GPAT3 activity and/or expression either directly or indirectly, including antibodies and antibody fragments that bind GPAT3 fragments); and agonist small molecules). Several pharmacogenomic approaches to consider in determining whether to administer a GPAT3 agonist or antagonist are well known to one of skill in the art and include genome-wide association, candidate gene approach, and gene expression profiling. A pharmaceutical composition of the invention is formulated to be compatible with its intended route of administration (e.g., oral compositions generally include an inert diluent or an edible carrier). Other nonlimiting examples of routes of administration include parenteral (e.g., intravenous), intradermal, subcutaneous, oral (e.g., inhalation), transdermal (topical), transmucosal, and rectal administration. The pharmaceutical compositions compatible with each intended route are well known in the art.

A GPAT3 antagonist(s) or agonist(s) may be used as a pharmaceutical composition when combined with a pharmaceutically acceptable carrier. Such a composition may contain, in addition to a GPAT3 antagonist(s) or agonist(s) (e.g., a human GPAT3 antagonist or agonist), carriers, various diluents, fillers, salts, buffers, stabilizers, solubilizers, and other materials well known in the art. The term “pharmaceutically acceptable” means a nontoxic material that does not interfere with the effectiveness of the biological activity of the active ingredient(s). The characteristics of the carrier will depend on the route of administration.

The pharmaceutical composition of the invention may also contain additional therapeutic agents for treatment of the particular targeted disorder. For example, a pharmaceutical composition for treatment of type 2 diabetes may also include an antidiabetic drug. The pharmaceutical composition may contain thrombolytic or antithrombotic factors such as plasminogen activator and Factor VIII. The pharmaceutical composition may further contain anti-inflammatory agents. Such additional factors and/or agents may be included in the pharmaceutical composition to produce a synergistic effect with GPAT3 antagonist(s) or agonist(s), or to minimize side effects caused by the GPAT3 antagonist(s) or agonist(s).

The pharmaceutical composition of the invention may be in the form of a liposome in which a GPAT3 antagonist(s) or agonist(s) is combined, in addition to other pharmaceutically acceptable carriers, with amphipathic agents such as lipids that exist in aggregated form as micelles, insoluble monolayers, liquid crystals, or lamellar layers in aqueous solution. Suitable lipids for liposomal formulation include, without limitation, monoglycerides, diglycerides, sulfatides, lysolecithin, phospholipids, saponin, bile acids, etc.

As used herein, the term “therapeutically effective amount” means the total amount of each active component of the pharmaceutical composition or method that is sufficient to show a meaningful patient benefit, e.g., amelioration of symptoms of, healing of, or increase in rate of healing of such conditions. When applied to an individual active ingredient, administered alone, the term refers to that ingredient alone. When applied to a combination, the term refers to combined amounts of the active ingredients that result in the therapeutic effect, whether administered in combination, serially or simultaneously.

In practicing the method of treatment or use of the present invention, a therapeutically effective amount of a GPAT3 antagonist(s) or agonist(s) is administered to a subject, e.g., a mammal (e.g., a human). A GPAT3 antagonist(s) or agonist(s) may be administered in accordance with the method of the invention either alone or in combination with other therapies, such as, e.g., in combination with additional therapies for, e.g., obesity, type 2 diabetes, or lipodystrophy. When coadministered with one or more agents, a GPAT3 antagonist(s) or agonist(s) may be administered either simultaneously with the other agent, or sequentially. If administered sequentially, the attending physician will decide on the appropriate sequence of administering the GPAT3 antagonist(s) or agonist(s) in combination with other agents.

When a therapeutically effective amount of a GPAT3 antagonist(s) or agonist(s) is administered orally, the binding agent will be in the form of a tablet, capsule, powder, solution or elixir. When administered in tablet form, the pharmaceutical composition of the invention may additionally contain a solid carrier such as a gelatin or an adjuvant. When administered in liquid form, a liquid carrier such as water, petroleum, oils of animal or plant origin such as peanut oil (exercising caution in relation to peanut allergies), mineral oil, soybean oil, or sesame oil, or synthetic oils may be added. The liquid form of the pharmaceutical composition may further contain physiological saline solution, dextrose or other saccharide solution, or glycols such as ethylene glycol, propylene glycol, or polyethylene glycol.

When a therapeutically effective amount of a GPAT3 antagonist(s) or agonist(s) is administered by intravenous, cutaneous or subcutaneous injection, the GPAT3 antagonist(s) or agonist(s) will be in the form of a pyrogen-free, parenterally acceptable aqueous solution. A preferred pharmaceutical composition for intravenous, cutaneous, or subcutaneous injection should contain, in addition to the GPAT3 antagonist(s) or agonist(s), an isotonic vehicle such as sodium chloride injection, Ringer's injection, dextrose injection, dextrose and sodium chloride injection, lactated Ringer's injection, or other vehicle as known in the art.

The amount of a GPAT3 antagonist(s) or agonist(s) in the pharmaceutical composition of the present invention will depend upon the nature and severity of the condition being treated, and on the nature of prior treatments that the patient has undergone. Ultimately, the attending physician will decide the amount of GPAT3 antagonist(s) or agonist(s) with which to treat each individual patient. Initially, the attending physician will administer low doses of GPAT3 antagonist(s) or agonist(s) and observe the patient's response. Larger doses of GPAT3 antagonist(s) or agonist(s) may be administered until the optimal therapeutic effect is obtained for the patient, and at that point the dosage is not generally increased further.

The duration of intravenous (i.v.) therapy using a pharmaceutical composition of the present invention will vary, depending on the severity of the disease being treated and the condition and potential idiosyncratic response of each individual patient. Also contemplated is subcutaneous (s.c.) therapy using a pharmaceutical composition of the present invention. The attending physician will decide on the appropriate duration of i.v. or s.c. therapy, or therapy with a small molecule, and the timing of administration of the therapy, using the pharmaceutical composition of the present invention.

The polynucleotides and proteins of the present invention are expected to exhibit one or more of the uses or biological activities (including those associated with assays cited herein) identified below. Uses or activities described for proteins of the present invention may be provided by administration or use of such proteins or by administration or use of polynucleotides encoding such proteins (such as, for example, in gene therapies or vectors suitable for introduction of DNA).

Uses of GPAT3 Agonists and Antagonists

In one aspect, the invention features a method of regulating TAG levels in a cell or sample of interest (e.g., in a tissue such as heart or blood). One such method comprises contacting a cell or population of cells with a GPAT3 antagonist(s) or agonist(s) in an amount sufficient to modulate the level of TAG in the cell or sample of interest. In one embodiment of the invention, a GPAT3 agonist is used, such that the level of TAG is increased in the cell or sample of interest. In another embodiment of the invention, a GPAT3 antagonist is used, such that the level of TAG is decreased in the cell or sample of interest. Modulation of TAG levels is expected to be beneficial for individuals suffering from GPAT3-associated conditions, and/or conditions accompanied by TAG dysregulation.

In other embodiments of the invention, a GPAT3 agonist or antagonist is used to modulate levels of TAG precursors, i.e., PA, LPA, DAG, and/or G3P. Modulating levels of such TAG precursors is expected to be beneficial in several respects. For example, LPA influences the developing and adult cardiovascular system, reproductive system, immune system, and nervous system (Anliker and Chun (2004) J. Biol. Chem. 279:20555-58), and contributes to wound healing (Mazereeuw-Hautier et al. (2005) J. Invest. Dermatol. 125(3):421-27). PA is the precursor of phosphatidylinositol, phosphatidylglycerol and cardiolipin, phospholipids that are autoantibody targets in antiphospholipid syndrome (Ulcova-Gallova (2005) Chem. Immunol. Allergy. 88:139-49) and systemic lupus erythematosus (Rhaman (2004) Rheumatology (Oxford) 43(11):1326-36), while cardiolipin appears to play a role in X-linked cardioskeletal myopathy and neutropenia (Barth syndrome) (Barth et al. (2004) Am. J. Med. Genet. A. 126(4):349-54). PA is also an important messenger in a common signaling pathway activated by proinflammatory mediators such as IL-1, TNFα, platelet activating factor, and lipid A (Bursten et al. (1992) Am. J. Physiol. 262:C328; Bursten et al. (1991) J. Biol. Chem. 255:20732; Kester (1993) J. Cell Physiol. 156:317). PA has been implicated in mitogenesis of several cell lines, and is increased in either ras- or fps-transformed cell lines compared to the parental Rat2 fibroblast cell line (Martin et al. (1997) Oncogene 14:1571). Activation of Raf-1, which is initiated by association of the molecule with the intracellular membrane, is an essential component of the MAPK signaling cascade. More importantly, recruitment of Raf-1 to membranes is reported to be mediated by direct association with phosphatidic acid (Rizzo et al. (2000) J. Biol. Chem. 275:23911-18). Thus, regulators of cellular levels of PA may play a role in cancer, and/or mediate inflammatory responses to various proinflammatory agents.

Further, it is known that DAG, in addition to being a second messenger in a number of cellular events requiring protein kinase C (PKC) activity, is the precursor of the major phospholipids phosphatidylcholine (PC), phosphatidylethanolamine (PE), and phosphatidylserine (PS), which have roles in membrane biosynthesis and integrity, phospholipase activation, and apoptosis and cancer (Wright et al. (2004) Biochem. Cell Biol. 82:18-26; Jenkins and Froham (2005) Cell. Mol. Life Sci. 62:2305-16; Hanshaw and Smith (2005) Bioorg. Med. Chem. 13:5035-42). DAG/PKC activity is implicated in numerous pathological events, including hyperglycemia and endothelial cell dysfunction (see, e.g., Hink et al. (2003) Treat Endocrinol. 2:293-304), Alzheimer's disease (e.g., Rossner (2004) Int. J. Dev. Neurosci. 22:467-74), cancer (e.g., Geiger et al. (2003) Curr. Opin. Mol. Ther. 5:631-41), and other disorders (e.g., Kawakami et al. (2002) J. Biochem. (Tokyo) 132:677-82).

Agonists or antagonists of GPAT3 may also be administered to subjects for whom regulation of GPAT3 activity is desired. These subjects may be afflicted with a condition such as dyslipidemia (e.g., hyperlipidemia, hypertriglyceridemia, Type III hyperlipidemia), obesity, hypercholesterolemia, hepatic steatosis, cancer, skin disorders associated with altered lipid metabolism (e.g., acne vulgaris, dry skin), adiposity, type 2 diabetes (and complications associated therewith, such as dermopathy, retinopathy, neuropathy, and nephropathy), insulin resistance, hyperinsulinemia, hypertension, cardiovascular disease, atherosclerosis, stroke, thrombosis, lipodystrophy (including congenital generalized lipodystrophy (Berardinelli-Seip syndrome), familial partial lipodystrophy (Dunnigan type, Köbberling type, and the mandibuloacral dysplasia type), and acquired forms of lipodystrophy such as acquired generalized lipodystrophy (Lawrence syndrome), acquired partial lipodystrophy (Barraquer-Simons syndrome), and lipodystrophy induced by antiviral treatments, e.g., treatment with HIV protease inhibitors), lipopenia, Reye's syndrome, Cushing's syndrome, metabolic syndrome (e.g., syndrome X), eating disorders (e.g., anorexia, bulimia), skin homeostasis, disorders related to energy storage, nutrient absorption, and lipid metabolism, reduced or absent lactation, and low preterm birth weight (and complications thereof, such as defects in neural development).

These methods are based, at least in part, on the finding that GPAT3 expression results in increased production of TAG via G3P acylation. Accordingly, GPAT3 antagonists, i.e., molecules that inhibit GPAT3 activity (e.g., antagonist anti-GPAT3 antibodies) may be used to decrease TAG levels in vivo, e.g., for treating or preventing disorders related to increased TAG synthesis or accumulation, such as obesity. Further, GPAT3 agonists, i.e., molecules that enhance GPAT3 activity (e.g., agonist anti-GPAT3 antibodies) may be used to increase TAG levels in vivo, e.g., for treating or preventing disorders related to decreased TAG synthesis or accumulation, such as lipodystrophy.

By using a GPAT3 agonist(s) and antagonist(s), it is possible to modulate TAG synthesis and accumulation in a number of ways. For example, decreasing TAG synthesis and/or accumulation (and/or accumulation of TAG precursors, i.e., DAG, LPA, PA, or G3P) may be in the form of inhibiting or blocking an established GPAT3-associated condition or disorder, or may involve preventing the induction of a GPAT3-associated conditions or disorders.

In one embodiment, a GPAT3 agonist(s) or antagonist(s), including pharmaceutical compositions thereof, is administered in combination therapy, i.e., combined with other agents, e.g., therapeutic agents, that are useful for treating pathological conditions or disorders, such as disorders of lipid metabolism or the cardiovascular system. The term “in combination” in this context means that the agents are given substantially contemporaneously, either simultaneously or sequentially. If given sequentially, at the onset of administration of the second compound, the first of the two compounds is preferably still detectable at effective concentrations at the site of treatment.

Preferred therapeutic agents used in combination with a GPAT3 agonist(s) or antagonist(s) are those agents that modulate different stages of TAG synthesis, e.g., agents that interfere with the activity of AGPAT, PTP, or DGAT, as well as agents that increase fatty acid utilization, such as PPARα and 6 modulators. Thus, agents useful in combination with a GPAT3 antagonist(s) or agonist(s) include, without limitation, PPARY modulators (e.g., glitazones, fatty acids (including polyunsaturated fatty acids)), PPARα modulators (e.g., fibrates (such as clofibrate, gemfibrozol, and Wy-14,643)), PPAR6 modulators, eicosapentaenoic acid, xanthohumols, roselipins, prenylflavonoids, polyacetylenes, tanshinones and derivatives thereof (see Coleman and Lee (2004), supra; Chen and Farse (2005), supra; Rustan et al. (1 988) J. Lipid Res. 29:1417-26; Tabata et al. (1997) Phytochemistry 46:683-87; Tomoda (1 999) J. Antibot. 52:689-94; Chung et al. (2004) Planta Med. 70:258-60; Lee et al. (2004) Planta Med. 70:197-200; Ko et al. (2002) Arch. Pharm. Res. 25:446-48); inhibitors of cholesterol acyltransferase enzymes (see Krause et al. (1995) Inflammation Mediators and Pathways, pp. 173-98 CRC Press, Boca Raton, Fla.); agents for the treatment of diabetes (e.g., insulin, insulin sensitizers such as metformin; Glp-1 mimetics, such as exenatide (BYETTA®); insulin secretagogues, such as sulfonylureas (e.g., tolazamide, glyburide and others) and metiglinides (e.g., nateglinide (STARLIX®)); modulators of sterol regulatory element-binding protein (SREBP), such as atorvastatin and simvastatin (e.g., LIPITOR® and CADUET®); modulators of liver X receptors (LXR) (e.g., oxysterols) and farnesoid X receptor (FXR) (e.g., bile acids); and other modulators of tissue lipid and cholesterol levels.

Another aspect of the present invention accordingly relates to kits for carrying out the administration of a GPAT3 agonist(s) or antagonist(s) with other therapeutic compounds. In one embodiment, the kit comprises one or more GPAT3 agonists or antagonists (e.g., one or more GPAT3 antagonists) formulated with one or more binding agents in a pharmaceutical carrier, and at least one other agent, e.g., another therapeutic agent, formulated as appropriate, in one or more separate pharmaceutical preparations. Kits related to diagnostic methods, prognostic methods, monitoring methods, etc., are also contemplated.

The entire contents of all references, patents, and patent applications cited throughout this application are hereby incorporated by reference herein.

### EXAMPLES

The following Examples provide illustrative embodiments of the invention and do not in any way limit the invention. One of ordinary skill in the art will recognize that numerous other embodiments are encompassed within the scope of the invention.

The Examples do not include detailed descriptions of conventional methods, such methods employed in the construction of vectors, the insertion of genes encoding polypeptides into such vectors and plasmids, the introduction of such vectors and plasmids into host cells, and the expression of polypeptides from such vectors and plasmids in host cells. Such methods are well known to those of ordinary skill in the art.

### Example 1

### Identification of GPAT3 as a Candidate for Microsomal GPAT

To identify genes that might encode proteins with microsomal GPAT activity, the following criteria were employed: 1) the gene should be a member of the acyltransferase family of proteins; 2) the mRNA should be abundantly expressed in tissues where glycerolipids are actively metabolized; 3) the mRNA should be upregulated during 3T3-LI adipocyte differentiation (Yet et al., supra; Coleman et al. (1978), supra); and 4) the calculated molecular mass should be about 45 kDa based on a previous purification study (Mishra and Kamisaka, supra). Using a seed alignment sequence derived from the previously described glycerolipid acyltransferase motif (PF01553) (Coleman and Lee, supra), a sequence homology search of public databases was performed. A profile hidden Markov model (profile HMM) was generated with the glycerophosphate acyltransferase model (PFO1553) in Pfam conserved domain database (Finn et al. (2006) Nucleic Acids Res. 34:D247-51), as well as with recently reported yeast endoplasmic reticulum-bound GPATs (Zheng and Zou, supra). The built HMM profile was then queried against SwissPort/TrEMBL (Boeckmann et al. (2003) Nucleic Acids Res. 31:365-70), RefSeq (Pruitt et al. (2005) Nucleic Acids Res. 33:D501-04), Ensembl (Birney et al. (2006) Nucleic Acids Res. 34:D556-61) and mouse and human genome databases to retrieve a list of sequences containing the glycerophosphate acyltransferase domain. A nonredundant set of such sequences was further generated by filtering out duplicate and alternative splice variants.

The database search identified a large number of candidates for glycerolipid acyltransferases, which included proteins with known functions, such as mitochondrial GPAT1, GNPAT, AGPAT1 and 2, ALCAT1, LPGAT1, and many hypothetical proteins of unknown functions. Comparison of the two datasets with transcription profiling data identified a previously uncharacterized mouse gene (accession number NM 172715), predicted to encode a 49.9-kDa protein. Based on data from Affymetrix gene chip analysis, the mRNA of the NM_{—}172715 gene was most abundant in white adipose tissue, and was 60-fold upregulated during 3T3-LI preadipocytes differentiation. A closely related human gene, MGC 11324 (accession number NM_{—}032717), was also identified. These genes are designated in the present study as mouse and human GPAT3 (mGPAT3 and hGPAT3). The mouse and human GPAT3 genes encode 438- and 434-amino acid proteins, respectively, which share 95% identity (FIG. 1A). Both protein sequences are predicted to be integral membrane proteins with at least two transmembrane domains (FIG. 1A), and both contain all four conserved acyltransferase motifs within a 133-amino acid region, as revealed by an alignment with hGPAT1 and hAGPAT1 (FIG. 1B). Furthermore, both proteins are predicted to be localized to the endoplasmic reticulum using multiple prediction algorithms including PSORT (data not shown) (Nakai and Horton (1999) Trends Biochem. Sci. 24:34-36), MITOPROT (Claros and Vincens (1996) Eur. J. Biochem. 241:779-86), PREDOTAR (Small et al. (2004) Proteomics 4:1581-90), and TargetP (Emanuelsson et al. (2000) J. Mol. Biol. 300:1005-16). Despite the presence of the acyltransferase motifs, human and mouse GPAT3 have less than 15% sequence identity with mtGPAT1 and previously identified yeast and plant GPATs (data not shown) (Zheng et al. (2003) Plant Cell 15:1872-87; Zheng and Zou (2001) J. Biol. Chem. 276:41710-16).

### Example 2

### GPAT3 Expressed in Sf9 Cells Possesses GPAT Activity

To determine whether the newly identified genes encode proteins with GPAT activity, native and N-terminally FLAG-tagged mouse and human GPAT3 were overexpressed in Sf9 cells. Briefly, full-length mouse and human GPAT3 were cloned by PCR amplification from cDNA libraries from mouse 17-day embryo and human leukocytes (BD Biosciences, San Diego, Calif.) using the following primers (5′ to 3′):

mGPAT3 forward (SEQ ID NO:5)
cgtgctgagacatggagggcgc;
mGPAT3 reverse (SEQ ID NO:6)
agccatgtttatccacgatgct;
hGPAT3 forward (SEQ ID NO:7)
ctcctgagtgggtgcgccgagt;
and
hGPAT3 reverse (SEQ ID NO:8)
tgtcatccgtcctcttagctga.

The PCR amplification was performed using a PHUSION™ DNA polymerase (Finnzymes, Finland) and the following thermal cycling conditions: initial denaturation at 98° C. for 60 s, then 35 cycles of denaturing at 98° C. for 10 s, annealing at 61° C. for 20 s, and extension at 72° C. for 30 s, followed by 72° C. for 5 min. PCR products were cloned into pPCR-SCRIPT® Amp SK(+) vector (Stratagene, San Diego, Calif.) and sequenced. To facilitate the detection of recombinant GPAT3 protein, N-terminal FLAG-tagged mGPAT3 and hGPAT3 were also engineered and cloned into the pPCR-SCRIPT(g Amp SK(+) vector by PCR using the following forward primers (5′ to 3′):

FLAG-mGPAT3 (SEQ ID NO:9):
ccaccatggactacaaagacgatgacgacaaggagggcgcagacctggcg
gtg;
and
FLAG-hGPAT3 (SEQ ID NO:10):
ccaccatggactacaaagacgatgacgacaaggagggcgcagagctggcc
ggg.

The reverse primers were the same as for untagged versions. For expression in insect cells, mouse and human cDNAs (with or without FLAG tag) were subcloned into the pFASTBAC™TM1 vector (Invitrogen, Carlsbad, Calif.). An N-terminally FLAG-tagged human DGAT1 clone was generated as previously described (Cases et al., supra). Recombinant baculovirus was generated using the BAC-TO-BAC® baculovirus expression system (Invitrogen, Carlsbad, Calif.).

Sf9 cells were infected with recombinant baculovirus at an MOI of 10 for 64 hrs. Cell pellets were harvested in ice-cold phosphate-buffered saline (PBS), lysed by sonication or Parr bomb, and total lysate was used immediately for enzyme assay. For subcellular fractionation, cel.ls were lysed with a rapid nitrogen decompression method (Parr Instrument Company, IL), followed by differential centrifugation at 8,000g (mitochondrial fraction) and 1 00,000g (microsomal fraction). Total protein concentration was assayed using a Bio-Rad protein assay with bovine serum albumin (BSA) as a standard (Bio-Rad, Hercules, Calif.). GPAT activity was determined by measuring the conversion of glycerol 3-phosphate (G3 P) to 1-acyl-sn-glycerol 3-phosphate in the presence of acyl-CoA. Formation of enzymatic products was detected by either the conventional 1-butanol extraction method followed by scintillation counting, or by thin layer chromatography (TLC) separation followed by exposure to a phosphorimager screen. GPAT activity assay by I -butanol extraction was performed as described (Yet et al., supra; Haldar and Vancura (1992) Methods Enzymol. 209:64-72). Briefly, cell lysates containing 100 μg protein were incubated for 20 min at room temperature in 75 mM Tris HCl, pH 7.5, 4 mM MgCl₂, 1 mg/ml fatty acid-free BSA, 8 mM NaF, 50 μM lauroyl-CoA, 3 mM glycerol 3-phosphate, and 1 μCi of [³H]glycerol 3-phosphate (30 Ci/mmol, American Radiolabeled Chemicals, Inc., St. Louis, Mo.) in a total volume of 250 μl. The reaction was stopped by addition of 0.5 ml of water-saturated 1-butanol and 0.5 ml of 1-butanol-saturated water followed by a vigorous vortex for 5 min. After a brief spin, the top phase (butanol) was transferred to a fresh tube and washed again with 0.5 ml of butanol-saturated water. Finally, an aliquot of the butanol phase was mixed with scintillation cocktail to count radioactivity using Accurate Radioisotope Counting (ARC).

To assess the GPAT activity more precisely, a TLC separation procedure was utilized. Briefly, the reaction was conducted in a total volume of 100 μl in the same reaction buffer as described above with: 1) 100 μM glycerol 3-phosphate, 50μM [¹⁴C]glycerol 3-phosphate (American Radiolabeled Chemicals, St. Louis, Mo., 55 mCi/mmol) and 50 μM lauroyl-CoA; or 2) 25 μM [¹⁴C]lauroyl-CoA and 0.5 mM glycerol 3-phosphate. To determine sensitivity to N-ethylmaleimide (NEM), cell lysates were incubated for 15 min in the presence or absence of 0.4 μM NEM prior to the initiation of reaction. Lipids were extracted using I ml of chloroform:methanol (2: 1, v/v), dried, and separated by TLC with chloroform:methanol:water (65:25:4, v/v). After separation, TLC plates were exposed to a phosphorimager screen to visualize the radiolabeled products with a Bio-Rad scanner (Hercules, Calif.). Acyltransferase activity towards various phospholipids or neutral lipids, such as lysophosphatidic acid (LPA), lysophosphatidylcholine (LPC), lysophosphatidylserine (LPS), lysophosphatidylglycerol (LPG), monoacylglycerol (MAG), and diacylglycerol (DAG), was assayed as described (Cao et al. (2004), supra; Cao et al. (2003), supra; Yang et al., supra) using 25 μM [¹⁴C]lauroyl-CoA as acyl donor. For experiments analyzing acyl-CoA donor preference (FIG. 2F), several radiolabeled acyl-CoA species were used, including palmitoyl-CoA (C16:0), oleoyl-CoA (C18:1), linoleoyl-CoA (C18:2), arachidoyl-CoA (C20:0), and arachidonoyl-CoA (C20:4). For TLC, enzymatic products were identified using standards detected by exposure to 12 vapor. All quantitative data are expressed as mean±S.E. Statistical analyses for differences between two groups were carried out using a Student's t-test.

Western analysis confirmed expression of mouse and human GPAT3 proteins (FIG. 2A). FLAG-tagged mouse and human GPAT3 migrated to positions corresponding to an apparent molecular mass of 50 kDa (for both proteins), consistent with the predicted molecular weights of 49.9 and 49.5 kDa. GPAT activity was initially assessed in lysates from cells overexpressing GPAT3 using a conventional extraction assay that measures incorporation of [³H]G3P into butanol-extractable products (Yet et al., supra; Haldar and Vancura, supra). Lauroyl-CoA was initially used as an as acyl donor, because recombinant mtGPAT (GPAT1) showed a preference for lauroyl-CoA over longer acyl-CoA species (data not shown). Lysates from Sf-9 cells overexpressing mGPAT3 or hGPAT3 showed a significant increase in the formation of butanol-extractable radiolabeled lipids, as compared to wild type cells or cells overexpressing hDGAT1 (FIG. 2B). To directly demonstrate formation of LPA, the product of the GPAT reaction, lipids were separated by TLC. Using [¹⁴C]G3P as the radiolabeled substrate (FIG. 2C, left panel), the formation of LPA was enhanced 2.5- and 4.2-fold in lysates from cells overexpressing mGPAT3 and hGPAT3, respectively. Similarly, using [¹⁴C]lauroyl-CoA as the radiolabeled substrate (FIG. 2C, right panel), formation of LPA was increased 1.8- and 3.0-fold, respectively. No LPA formation was observed in samples lacking either one of the substrates (note “-Acyl-CoA” and “- G3P” lanes in FIG. 2C). TLC separation also showed the presence of several non-LPA lipids derived from either [¹⁴C]G3P or [¹⁴C]lauroyl-CoA (e.g., upper bands in FIG. 2C), which may represent products of endogenous enzymes in insect cells. The presence of these non-LPA lipids in the butanol extract likely accounts for the higher background and decreased sensitivity of this method. Thus, GPAT activity was assessed by TLC separation in all subsequent experiments. Formation of LPA increased in a substrate concentration-dependent manner for both [¹⁴C]G3P and lauroyl-CoA, and was significantly higher in hGPAT3-expressing cells compared to controls at all substrate concentrations tested (FIG. 2D). hGPAT3-dependent LPA formation decreased at very high concentrations of acyl-CoA (FIG. 2D), similar to what has been reported for mtGPAT1 (Yet et al. (1993) Biochemistry 32:9486-91). The maximal hGPAT3-dependent increase in LPA formation corresponded to ˜100 pmol/min/mg protein with half-maximal activity reached at ˜25 μM lauroyl-CoA and ˜80 μM G3P (FIG. 2D). This assay was performed using a nonpurified system; variations in activity and fold-increase (2.5-10-fold; average 6-fold) between experiments likely reflect differences in expression levels between different preparations. Upon hGPAT3 overexpression in HEK293 cells, there was a similar, although on average less pronounced, increase in LPA formation compared to control cells (˜3-fold increase; ˜100 pmol/min/mg) (FIG. 2E, right panel). Importantly, the fold-increase observed upon GPAT3 overexpression was comparable to the one observed with mtGPAT1 (2.3- versus 3.8-fold; FIG. 2E, right panel).

Microsomal GPAT activity has been shown to be sensitive to NEM (Coleman and Lee, supra). Pretreatment of either Sf-9 or HEK293 lysates with NEM completely abolished the increase in LPA formation conferred by hGPAT3 overexpression (FIG. 2E), while mtGPAT1-dependent LPA formation was not affected by NEM (FIG. 2E, right panel). Sequence alignment of GPAT3 orthologs from different species revealed several conserved cysteine residues not shared by mtGPAT1 that may account for the NEM-sensitivity of GPAT3 (data not shown). The ability of hGPAT3 to utilize different fatty acyl-CoA species as substrates was also tested. An hGPAT3-dependent increase in LPA formation was observed for all acyl-CoA species examined, indicating that hGPAT3 can utilize a broad range of long chain fatty acyl-CoA, both saturated and unsaturated species (FIG. 2F). Experiments were also conducted to test whether hGPAT3 can utilize acyl acceptors other than glycerol 3-phosphate. As shown in FIG. 2G, no significant increase in product formation was observed when LPA, lysophosphatidylcholine (LPC), lysophosphatidylserine (LPS), lysophosphatidylglycerol (LPG), monoacylglycerol (MAG) or diacylglycerol (DAG) were presented as substrates (FIG. 2G).

### Example 3

### Ectopic Expression of GPAT3 Increases TAG Formation

To further investigate the role of GPAT3 in TAG and/or phospholipid synthesis in mammalian cells, GPAT3 was overexpressed in HEK293 cells and the incorporation of [¹⁴C]oleic acid into TAG or phospholipids was measured. Briefly, mouse and human GPAT3 cDNAs (with or without FLAG tag) were subcloned into pcDNA3. 1 (+)/Hygro. DGAT 1 and GPAT 1 cDNAs were cloned as described (Cao et al. (2004), supra; Cases et al., supra). DNA was transfected into cells using FUGENE®6 according to the manufacturer's instruction (Roche Diagnostics, Nutley, N.J.).

HEK293 cells were transfected with empty pcDNA3.1 vector (control) or vectors containing mGPAT3, hGPAT3, hGPAT1, or hDGAT1 (see above). Forty hrs after transfection, cells were incubated with 2 μM of [¹⁴C]oleic acid (50 Ci/mmol) in medium supplemented with 0.1% fatty acid free BSA for 6 hrs. At the end of incubation, cells were washed twice with cold PBS and collected. Total lipids were extracted with chloroform/methanol (2:1, v/v), dried, and resolved by TLC with chloroform:methanol:water (65:25:4, v/v, polar lipids) and hexane:ethyl ether:acetic acid (80:20:1, v/v/v, neutral lipids). Radiolabeled triacylglycerols or phospholipids were quantitated by exposure to a phosphorimager screen.

As shown in FIG. 3A, cells overexpressing mGPAT3 and hGPAT3 incorporated significantly more radiolabeled oleic acid into TAG compared with cells transfected with empty vector (Control). Formation of labeled TAG was significantly increased (˜3 to 4-fold) in GPAT3-overexpressing cells compared to control cells (FIG. 3A), while no increase in phospholipid formation was observed (FIG. 3B). Overexpression of GFP or other proteins such as adiponutrin did not increase TAG synthesis under the same experimental conditions (data not shown). The increase in TAG formation upon human or mouse GPAT3 (hGPAT3 and mGPAT3, respectively) overexpression was similar in magnitude to the increases observed upon overexpression of hDGAT1 or mtGPAT (hGPAT1) (FIG. 3A).

### Example 4

### GPAT3 Localizes in the Endoplasmic Reticulum

To define the subcellular localization of GPAT3, indirect immunofluorescence on COS-7 cells transiently transfected with FLAG-tagged mGPAT3 was performed. Briefly, COS-7 cells were grown and transfected on a cover slip (BD Biosciences, Bedford, Mass.) with empty pcDNA3.1 vector (control) or a vector containing mGPAT3, as in a procedure previously described (Cao et al. (2004), supra). Forty-eight hrs after transfection, mitochondria were stained by incubating cells with 100 nM MITOTACKER® Red CMXRos (Invitrogen, Carlsbad, Calif.) for 30 min at 37° C. in growth medium. Cells were fixed with 4.0% paraformaldehyde and permeabilized with 0.2% Triton X-100 in PBS. After being rinsed with PBS twice for 5 min each, cells were incubated in 5% normal donkey serum in PBS for 1 h to block nonspecific binding. Samples were then incubated with mouse monoclonal anti-FLAG M2 antibody (5.0 pg/ml, Sigma, St. Louis, Mo.) or rabbit anti-calnexin (a resident ER transmembrane protein) amino-terminal polyclonal antibody (1.0 μg/ml, StressGen Biotechnologies Corp., Victoria, Canada) for 2 h at room temperature. After being washed with PBS three times for 5 min each, cells were incubated with Cy2-conjugated donkey anti-mouse IgG and Cy3-conjugated donkey anti-rabbit IgG (Jackson ImmunoResearch Laboratories Inc., West Grove, Pa.) for 1 h, washed four times with PBS, and analyzed in a Bio-Rad MRC-600® Laser Confocal Microscope System (Hercules, Calif.).

An immunohistochemical study was performed in COS-7 cells to examine the subcellular localization of GPAT3. By immunofluorescence, FLAG-mGPAT3 displayed a perinuclear pattern that was distinct from the staining of a mitochondrial marker (MitoTracker Red CMXRos), but colocalized well with an ER marker (Calnexin) (data not shown). While the calnexin/GPAT3-staining was reticular in some cells, the cells exhibiting the brightest GPAT3 staining showed the presence of Calnexin/GPAT3-labeled cisternae, possibly reflecting an effect of GPAT3 overexpression on ER morphology. Similar results were achieved with hGPAT3 (data not shown). To further investigate the subcellular localization of GPAT3, subcellular fractions enriched for mitochondria and microsomes were generated using differential sedimentation of HEK293 cell membranes overexpressing FLAG-tagged hGPAT3 or untagged mtGPAT1. Western blotting showed enrichment of the mitochondrial marker prohibitin in the mitochondrial fraction, while the ER marker calnexin was enriched in the microsomal fraction (FIG. 4A). FLAG-tagged hGPAT3 showed a fractionation pattern similar to calnexin (FIG. 4A), consistent with ER localization. The increase in GPAT activity in lysates from hGPAT3- and mtGPAT1-overexpressing cells was similar (˜2-fold compared to control, FIGS. 4B and 4C). mtGPAT1-overexpressing cells showed the largest increase in activity in the mitochondrial fraction, while GPAT activity was increased primarily in the microsomal fraction in hGPAT3-overexpressing cells (FIGS. 4B and 4C). These findings are consistent with GPAT3 being an ER-localized enzyme.

### Example 5

### Tissue Distribution of GPAT3 mRNA in Mouse and Human

To confirm and extend the transcriptional profiling data, TAQMAN® (Applied Biosystems, Foster City, Calif.) real-time quantitative PCR (Q-PCR) analysis was performed using probes specific for mouse and human GPAT3. Briefly, undifferentiated and differentiated 3T3-L1 adipocytes, tissues from normal 8-12 week old male C57B1/6J mice, and tissues from 10-week old male ob/ob and age-matched wild type control mice were obtained as previously described (Lake et al., supra). For PPARγ agonist treatment, 10-week old male ob/ob mice were gavaged once a day with 15 mg/kg rosiglitazone or vehicle control for 21 days. RNA and cDNA were prepared as previously described (Lake et al., supra). Gene profiling data were generated using the MOE430 chip according to the manufacturer's recommendations (Affymetrix, Santa Clara, Calif.). Q-PCR was performed using an ABI PRISM® 7900 sequence detector (Applied Biosystems, Foster City, Calif.) with 18s as an internal control as described (Lake et al., supra). Gene-specific primers and probes were obtained from Applied Biosystems (Cat. Nos. mm00554802_ml and Hs00262010_ml). Relative expression was determined by the C₁ method (Applied Biosystems, Foster City, Calif.).

Of the tissues examined, mGPAT3 mRNA was most abundant in epididymal fat (Ed. Fat), followed by small intestine (Sm. Intestine), brown adipose tissue (BAT), kidney, heart, and colon (FIG. 5A). In humans, GPAT3 mRNA was most highly expressed in kidney, heart, skeletal muscle, thyroid gland and testis. Significant levels were also found in lung and adipose tissue (FIG. 5B). No major alternative splice variants of mGPAT3 and hGPAT3 genes were found by database searching or Northern blot analysis (data not shown). Interestingly, the level of GPAT3 mRNA in both mouse and human liver was low, possibly suggesting the existence of additional genes that encode liver microsomal GPAT activity. With the exception of small intestine, liver and lung, mGPAT3 shows a tissue distribution similar to mouse mtGPAT1, while human mtGPAT1 is strikingly abundant in adipose tissue (FIG. 8).

Microsomal GPAT activity has previously been shown to significantly increased (˜70-fold) during differentiation of 3T3-L1 preadipocytes to adipocytes (Yet et al., supra; Coleman et al. (1978), supra). Disclosed herein is the finding that mGPAT3 mRNA is increased ˜60-fold in 3T3-L1 adipocytes compared to preadipocytes (FIG. 6A), concomitant with a similar increase in GPAT activity (data not shown).

To determine the contribution of GPAT3 to total GPAT activity in 3T3-L1 adipocytes, differentiated adipocytes were exposed to siRNA directed against GPAT3. Briefly, 3T3-L1 fibroblast cells were grown, maintained, and induced to differentiate into adipocytes as described (Cao et al., supra). On day 10 after differentiation, 2.5×10⁶ 3T3-L1 adipocytes suspended in 0.5 ml of PBS were transfected with 15 nmol of control siRNA (Ambion, Austin, Tex., Cat#4613) or mouse GPAT3-specific siRNA duplex (Ambion, Austin, Tex., Cat #16708A, siRNA ID 16316, 16317 [shown in FIGS. 6B and 6C], and 16318) by electroporation as described (Jiang et al. (2003) Proc. Natl. Acad. Sci. U.S.A. 100:7569-74). The mouse GPAT3 siRNA duplex targets exon 3 (encoding amino acids 59 to 116). After electroporation, cells were immediately mixed with fresh medium and incubated for 10 min on ice before seeding into 6-well plates. 72 h after electroporation, GPAT3 mRNA was measured by Q-PCR, and GPAT activity was determined as described above.

The results from the siRNA knockdown experiments are shown in FIGS. 6B and 6C. Depletion of mGPAT3 in differentiated 3T3-LI adipocytes using RNAi oligonucleotides resulted in a decrease in mGPAT3 mRNA by ˜60% (FIG. 6B), and a concomitant decrease in GPAT activity by ˜55% (FIG. 6C), compared to cells transfected with nontargeting control RNAi oligonucleotides. A significant decrease in GPAT3 mRNA, as well as GPAT3 activity, was also observed with two additional siRNA oligonucleotides targeting GPAT3 (data not shown). These data suggest that a significant part of the GPAT activity in differentiated 3T3-LI adipocytes is due to GPAT3 expression.

To further explore a role for GPAT3 in lipogenesis, GPAT3 expression was examined in ob/ob mice, a genetic model of obesity. mGPAT3 mRNA was significantly (70%) decreased in adipose tissue (FIG. 7A) and significantly increased (2-fold) in liver (FIG. 7B) of ob/ob mice. PPARγ agonists such as rosiglitazone induce expression of the lipogenic program in adipose tissue (Rosen and Spiegelman (2001) J. Biol. Chem. 276:37731-34). To examine the effect of rosiglitazone on GPAT3 activity, 10-week old male ob/ob mice were gavaged once a day with 15 mg/kg rosiglitazone or vehicle control for 21 days. As shown in FIG. 7C, treatment of ob/ob mice with rosiglitazone (Rosi) induced expression of mGPAT3 mRNA in white adipose tissue 4.5-fold over control. mtGPAT1 and DGAT1 mRNA also showed a large increase during 3T3-L1 differentiation (Yet et al., supra; Lake et al., supra) (FIGS. 9A and 9E) and a significant downregulation in adipose tissue of ob/ob mice (FIGS. 9B and 9F). The downregulation of mRNA for DGAT1 and other genes involved in lipogenesis in ob/ob white adipose tissue has been previously reported and has been attributed to dedifferentiation of adipose tissue in ob/ob mice (Suzuki et al. (2005) J. Biol. Chem. 280:3331-37; Nadler et al. (2000) Proc. Natl. Acad. Sci. USA. 97:11371-76). In contrast to GPAT3, the expression levels of mtGPAT1 and DGAT1 did not change in the liver of ob/ob mice, or in adipose tissue upon rosiglitazone treatment (FIGS. 9C and 9G, and FIGS. 9D and 9H, respectively). Taken together, the regulation of GPAT3 mRNA by rosiglitazone supports a role for this enzyme in lipogenesis.

What is claimed is:

1. A method for treating, ameliorating, or preventing a GPAT3-associated condition in a mammal comprising administering to the mammal a therapeutically effective amount of an agent that modulates the level of expression or activity of GPAT3 in the mammal.

2. The method as set forth in claim 1, wherein the agent is selected from the group consisting of GPAT3 inhibitory polynucleotides or fragments thereof, GPAT3 inhibitory polypeptides or fragments thereof, antagonistic anti-GPAT3 antibodies, antagonistic anti-GPAT3 antibody fragments, and small molecules.

3. The method as set forth in claim 1, wherein the agent is selected from the group consisting of GPAT3 polynucleotides or fragments thereof, polynucleotides that hybridize under high stringency conditions to a complement of the nucleic acid sequence or a fragment of the nucleic acid sequence as set forth in SEQ ID NO:1 or SEQ ID NO:3, GPAT3 polypeptides or fragments thereof, polypeptides encoded by a nucleic acid sequence or a fragment of a nucleic acid sequence as set forth in SEQ ID NO:1 or SEQ ID NO:3, polypeptides encoded by a nucleic acid that hybridizes under high stringency conditions to a complement of the nucleic acid sequence or a fragment of the nucleic acid sequence as set forth in SEQ ID NO:1 or SEQ ID NO:3, agonistic anti-GPAT3 antibodies, agonistic anti-GPAT3 antibody fragments, and small molecules.

4. A method for decreasing TAG synthesis and/or PA, LPA and/or DAG synthesis and/or accumulation in a cell or cell population, comprising contacting the cell or cell population with a GPAT3 antagonist in an amount sufficient to decrease the level of expression or activity of GPAT3 in the cell or cell population, wherein the GPAT3 antagonist is selected from the group consisting of GPAT3 inhibitory polynucleotides or fragments thereof, GPAT3 inhibitory polypeptides or fragments thereof, antagonistic anti-GPAT3 antibodies, antagonistic anti-GPAT3 antibody fragments, and small molecules.

5. A method for increasing TAG synthesis and/or PA, LPA and/or DAG synthesis and/or accumulation in a cell or cell population, comprising contacting the cell or cell population with a GPAT3 agonist in an amount sufficient to increase the level of expression or activity of GPAT3 in the cell or cell population, wherein the GPAT3 agonist is selected from the group consisting of GPAT3 polynucleotides or fragments thereof, polynucleotides that hybridize under high stringency conditions to a complement of the nucleic acid sequence or a fragment of the nucleic acid sequence as set forth in SEQ ID NO:1 or SEQ ID NO:3, GPAT3 polypeptides or fragments thereof, polypeptides encoded by a nucleic acid sequence or a fragment of a nucleic acid sequence as set forth in SEQ ID NO:1 or SEQ ID NO:3, polypeptides encoded by a nucleic acid that hybridizes under high stringency conditions to a complement of the nucleic acid sequence or a fragment of the nucleic acid sequence as set forth in SEQ ID NO:1 or SEQ ID NO:3, agonistic anti-GPAT3 antibodies, agonistic anti-GPAT3 antibody fragments, and small molecules.

6. A method for monitoring the course of a treatment of a GPAT3-associated condition in a patient, comprising:
(a) measuring the level of expression or activity of GPAT3 in a cell or sample of interest from the patient;
(b) administering a GPAT3 antagonist to the patient; and
(c) measuring the level of expression or activity of GPAT3 in a cell or sample of interest from the patient following administration of the GPAT3 antagonist,
wherein a lower level of expression or activity of GPAT3 in the cell or sample of interest from the patient following administration of the GPAT3 antagonist, in comparison to the level of expression or activity of GPAT3 in the cell or sample of interest from the patient prior to administration of the GPAT3 antagonist, provides a positive indication of the treatment of the GPAT3-associated condition in the patient.

7. A method for monitoring the course of a treatment of a GPAT3-associated condition in a patient, comprising:
(a) measuring the level of expression or activity of GPAT3 in a cell or sample of interest from the patient;
(b) administering a GPAT3 agonist to the patient; and
(c) measuring the level of expression or activity of GPAT3 in a cell or sample of interest from the patient following administration of the GPAT3 agonist,
wherein a greater level of expression or activity of GPAT3 in the cell or sample of interest from the patient following administration of the GPAT3 agonist, in comparison to the level of expression or activity of GPAT3 in the cell or sample of interest from the patient prior to administration of the GPAT3 agonist, provides a positive indication of the treatment of the GPAT3-associated condition in the patient.

8. A method for prognosing a GPAT3-associated condition in a patient, comprising:
(a) measuring the level of expression or activity of GPAT3 in a cell or sample of interest from the patient at a first time point; and
(b) measuring the level of expression or activity of GPAT3 in a cell or sample of interest from the patient at a second time point,
wherein a different level of expression or activity of GPAT3 in the cell or sample of interest from the patient at the second time point, in comparison to the level of expression or activity of GPAT3 in the cell or sample of interest from the patient at the first time point, indicates a decreased likelihood that the patient will develop a more severe form of the GPAT3-associated condition.

9. A method for prognosing a GPAT3-associated condition in a patient, comprising:
(a) measuring the level of expression or activity of GPAT3 in a cell or sample of interest from the patient; and
(b) comparing the level of expression or activity of GPAT3 in the cell or sample of interest to the level of expression or activity of GPAT3 in a reference cell or sample of interest,
wherein a different level of expression or activity of GPAT3 in the cell or sample of interest from the patient, in comparison to the level of expression or activity of GPAT3 in the reference cell or sample, indicates a decreased likelihood that the patient will develop a more severe form of the GPAT3-associated condition.

10. A method of screening for a compound capable of modulating GPAT3 activity comprising the steps of:
(a) contacting a sample containing GPAT3 with a compound of interest; and
(b) determining whether the level of expression or activity of GPAT3 in the contacted sample is modulated relative to the level of expression or activity of GPAT3 in a sample not contacted with the compound,
wherein a change in the level of expression or activity of GPAT3 in the contacted sample identifies the compound as a compound that is capable of modulating GPAT3 activity.

11. A pharmaceutical composition comprising a GPAT3 antagonist and a pharmaceutically acceptable carrier.

12. The pharmaceutical composition of claim 1 1, wherein the GPAT3 antagonist is selected from the group consisting of GPAT3 inhibitory polynucleotides or fragments thereof, GPAT3 inhibitory polypeptides or fragments thereof, antagonistic anti-GPAT3 antibodies, antagonistic anti-GPAT3 antibody fragments, and small molecules.

13. A pharmaceutical composition comprising a GPAT3 agonist and a pharmaceutically acceptable carrier.

14. The pharmaceutical composition of claim 13, wherein the GPAT3 agonist is selected from the group consisting of GPAT3 polynucleotides or fragments thereof, polynucleotides that hybridize under high stringency conditions to a complement of the nucleic acid sequence or a fragment of the nucleic acid sequence as set forth in SEQ ID NO:1 or SEQ ID NO:3, GPAT3 polypeptides or fragments thereof, polypeptides encoded by a nucleic acid sequence or a fragment of a nucleic acid sequence as set forth in SEQ ID NO:1 or SEQ ID NO:3, polypeptides encoded by a nucleic acid that hybridizes under high stringency conditions to a complement of the nucleic acid sequence or a fragment of the nucleic acid sequence as set forth in SEQ ID NO:1 or SEQ ID NO:3, agonistic anti-GPAT3 antibodies, agonistic anti-GPAT3 antibody fragments, and small molecules.

15. An antibody or antibody fragment that specifically binds a GPAT3 polypeptide or a fragment of a GPAT3 polypeptide.

16. The antibody or antibody fragment as set forth in claim 15, wherein the GPAT3 polypeptide is a mouse GPAT3 polypeptide or a human GPAT3 polypeptide.

17. The antibody or antibody fragment as set forth in claim 16, wherein the GPAT3 polypeptide comprises the amino acid sequence set forth in SEQ ID NO:2 or SEQ ID NO:4.

18. The antibody or antibody fragment as set forth in any one of claims 15-17, wherein the antibody antagonizes at least one GPAT3 activity.

19. The antibody or antibody fragment as set forth in any one of claims 15-17, wherein the antibody agonizes at least one GPAT3 activity.
